(19) United States
(12) Patent Application Publication
(10) Pub. No.: US 2020/0325521 A1
(43) Pub. Date: Oct. 15, 2020

(54) NUCLEIC ACID ENZYME-MEDIATED SIGNAL AMPLIFICATION FOR BIOSENSING

(71) Applicant: McMaster University, Hamilton (CA)

(72) Inventors: Yingfu Li, Dundas (CA); John Brennan, Dundas (CA); Meng Liu, Dundas (CA)

(21) Appl. No.: 16/492,262

(22) PCT Filed: Mar. 12, 2018
(86) PCT No.: PCT/CA2018/050294

§ 371 Date: Sep. 9, 2019

Related U.S. Application Data
(60) Provisional application No. 62/469,772, filed on Mar. 10, 2017.

(51) Int. Cl. C12Q 1/682; C12Q 1/6844; C12Q 1/689

(52) U.S. Cl. CPC C12Q 1/682; C12Q 2600/178; C12Q 1/689; C12Q 1/6844

(57) ABSTRACT

Described is an approach that takes advantage of rolling circle amplification (RCA) and an RNA-cleaving DNAzyme (RCD) to achieve massive signal amplification for biosensing via a cross-feedback mechanism. An RCA reaction generates copies of an RCD that triggers a reaction cascade designed to generate additional DNA assemblies for RCA. These cross-feedback actions work autonomously to turn limited molecular recognition events into massive amounts of DNA amplicons that can be conveniently detected. This approach was demonstrated for biosensing of a microRNA sequence and a bacterium.

### RELATED APPLICATIONS

The present application claims the benefit of U.S. provisional patent application No. 62/469,772 filed Mar. 10, 2017, the contents of which are herein incorporated by reference.

### FIELD

The present application relates to biosensors that detect analytes, and kits and methods of use thereof. In particular, the application relates to the use of RNA-cleaving DNA enzymes and rolling circle amplification for amplification of RCA templates.

### BACKGROUND

There is a growing need for highly sensitive biosensing methods that can enable early diagnosis of human diseases or early detection of harmful agents in food, water and the environment. However, standard biosensing methods based purely on receptor-ligand interactions are usually not sensitive enough to meet the challenge of early detection when disease biomarkers or environmental contaminants only exist at extremely low concentrations. Therefore, considerable efforts have been directed towards developing more powerful biosensing strategies that incorporate signal amplification mechanisms.

In vitro amplification of DNA represents a powerful signal amplification strategy owing to its capability to generate large quantities of DNA amplicons from a few input DNA molecules [1]. A particular DNA amplification technique, known as “rolling circle amplification (RCA)”, has attracted a great deal of attention in the field of biosensing because of its operational simplicity [2]. In this isothermal process, a special DNA polymerase, such as phi29 DNA polymerase (ϕ29DP), elongates a short DNA primer (DP) around a circular DNA template (CDT) round by round, generating long single-stranded (ss) DNA molecules with tandem sequence repeats [3]. Although standard RCA procedures produce DNA amplicons linearly, modified RCA methods capable of delivering exponential DNA amplification have also been reported [4,5]. However, most of these approaches are designed to detect only DNA or RNA as targets.

### SUMMARY

Although double-stranded (ds) DNA is widely known as genetic material, ssDNA has been shown to be able to function as both molecular receptors (DNA aptamers) and enzymes (DNAzymes) [6]. DNA aptamers and DNAzymes can be identified from random DNA pools via vitro selection [7]. One widely studied class of DNAzymes is RNA-cleaving DNAzymes (RCDs) [8]. A large number of RCDs have been reported [9] and many of them have been examined as biosensors for the detection of metal ions, small molecules and bacterial pathogens [10, 11].

Here, the inventors have demonstrated that RCDs can also be incorporated into RCA processes to create a feedback loop for autonomous DNA amplification. This approach has been termed “DNAzyme Feedback Amplification (DFA)”. The versatility of DFA for biosensing has been demonstrated through the design of DFA systems for ultra-sensitive detection of a microRNA and a model bacterial pathogen with sensitivity improvements of 3-6 orders of magnitude over conventional methods.

In one aspect, DFA takes advantage of rolling circle amplification (RCA) and an RNA-cleaving DNAzyme (RCD). For example, in one embodiment, DFA employs two specially programmed DNA complexes, one composed of a primer and a circular template containing the antisense sequence of an RCD, and the other composed of the same or a similar circular template and another primer that is also a RNA-containing substrate for the RCD. RCA is initiated on the first complex to produce RCD elements that go on to cleave the substrate in the second complex. This cleavage event triggers production of more input complexes for RCA. As shown in FIG. 1a, this reaction circuit continues autonomously, resulting in exponential DNA amplification.

In one embodiment the methods and products described herein use RCD and RCA to achieve signal amplification via a cross-feedback mechanism. In one embodiment, the approach begins with an RCA reaction that generates copies of an RCD and the production of RCD triggers a reaction cascade designed to generate additional DNA assemblies for RCA. These cross-feedback actions work autonomously to turn limited molecular recognition events into massive amounts of DNA amplicons that can be conveniently detected. Although the Examples illustrate the ultra-sensitive detection of microRNA and a bacterial pathogen, this approach can be used for many other analytes so long as the first RCA step can be effectively regulated by a molecular recognition event.

Accordingly, in one embodiment there is provided a method of detecting a target analyte in a sample. In one embodiment, the method comprises:

combining the sample with a first circular DNA template comprising a region encoding an antisense ribonucleotide-cleaving DNAzyme (RNA-cleaving DNAzyme), wherein the first circular DNA template is amplified by rolling circle amplification in the presence of the target analyte to produce a first amplification product comprising the RNA-cleaving DNAzyme;

contacting the first amplification product comprising the RNA-cleaving DNAzyme and a substrate complex comprising a ribonucleotide-containing DNA sequence (RDS) nucleic acid molecule annealed to a second circular DNA template, the second circular DNA template comprising a region encoding an antisense DNAzyme and a region complimentary to the 5′ end of the RDS nucleic acid molecule, wherein the RNA-cleaving DNAzyme acts on the substrate complex to produce a 5′ cleavage fragment comprising a 5′ region annealed to the second circular DNA template and a 3′ cleavage fragment;

amplifying the second circular DNA template by rolling circle amplification primed by a 3′-hydroxyl end of the 5′ region annealed to the second circular DNA template to produce a second amplification product comprising the DNAzyme; and

detecting an increase in the first amplification product and/or second amplification product thereby detecting the presence of the target analyte in the sample.

Different techniques may be used to initiate rolling circle amplification of the first circular DNA template in the presence of the target analyte. For example, in one embodiment, the target analyte is a target nucleic acid molecule that binds to the first circular DNA template and rolling circle amplification of the first circular DNA template is primed by a 3′-hydroxyl end of the target nucleic acid molecule that binds to the first circular DNA template. In another embodiment, the target analyte activates an exogenous RNA-cleaving DNAzyme that binds to a nucleic acid molecule annealed to the first circular DNA template comprising one or more RDS sequences to produce a 5′ cleavage product comprising a 5′ region annealed to the first circular DNA template, wherein rolling circle amplification of the first circular DNA template is primed by a 3′-hydroxyl end of the 5′ region annealed to the first circular DNA template. In a further embodiment, the nucleic acid molecule annealed to the first circular DNA template comprises a first RDS sequence that is cleaved by the exogenous RNA-cleaving DNAzyme and a second RDS sequence that is cleaved by an RNA-cleaving DNAzyme encoded by the second circular template, optionally wherein the exogenous RNA-cleaving DNAzyme is EC1. Optionally, the exogenous RNA-cleaving DNAzyme and the RNA-cleaving DNAzyme encoded by the second circular template cleave the same RDS sequence.

In another embodiment, the target analyte binds to a recognition moiety that directly or indirectly triggers rolling circle amplification of the first circular DNA template.

In one embodiment, the DNAzyme encoded by the second amplification product is an RNA-cleaving DNAzyme, optionally the same RNA-cleaving DNAzyme that is encoded by the first amplification product. In one embodiment, the second amplification product acts on the RDS nucleic acid molecule on the substrate complex to produce the 5′ cleavage fragment and the 3′ cleavage fragment. All or part of the 5′ cleavage fragment may then be used to prime rolling circle amplification of the second circular DNA template forming part of the substrate complex.

In one embodiment, the method comprises removing unpaired nucleotides from the 5′ cleavage fragment to form the 3′-hydroxyl end of the 5′ region annealed to the second circular DNA template. For example, in one embodiment the method comprises contacting the 5′-cleavage fragment with an enzyme having 3′-5′ exonuclease activity to trim or remove unpaired nucleotides. Optionally, the enzyme is a polymerase enzyme such as ϕ29DP that also has 5′-3′ polymerase activity.

In one embodiment, the methods described herein involve a ribonucleotide-containing DNA sequence (RDS) nucleic acid molecule, optionally wherein the RDS nucleic acid molecule is annealed to a circular DNA template. In one embodiment, a 5′ region of the RDS nucleic acid molecule comprises a sequence complementary to a sequence on the circular DNA template and a 3′ region of the RDS nucleic acid molecule comprises a sequence with at least one ribonucleotide e.g. a nucleotide containing ribose as its pentose component. The presence of the ribonucleotide renders the RDS nucleic acid molecule susceptible to cleavage by a ribonucleotide-cleaving DNAzyme. In one embodiment, the 3′ end of the RDS nucleic acid molecule is modified to prevent degradation of the single stranded 3′ region. This prevents rolling circle amplification of the circular DNA template prior to cleavage by the RNA-cleaving DNAzyme. For example, in one embodiment, the 3′ end of the RDS nucleic acid molecule comprises a 3′ Inverted dT leading to a 3′-3′ linkage which inhibits both degradation by 3′ exonucleases and extension by DNA polymerases.

In one embodiment, the methods described herein may be used to amplify a target sequence of a target nucleic acid molecule in a sample. In one embodiment, the method comprises:

combining the sample and a first circular DNA template comprising a region encoding an antisense ribonucleotide-cleaving DNAzyme (RNA-cleaving DNAzyme) and a region complimentary to the target sequence such that a 3′-hydroxyl end of the target nucleic acid molecule anneals to the region complimentary to the target sequence on the circular DNA template;

amplifying the first circular DNA template by rolling circle amplification primed by the 3′-hydroxyl end of the target nucleic acid molecule to produce a first amplification product comprising a RNA-cleaving DNAzyme, wherein the RNA-cleaving DNAzyme acts on a substrate complex comprising a ribonucleotide-containing DNA sequence (RDS) nucleic acid molecule annealed to a second circular DNA template to produce a 5′ cleavage fragment comprising a 5′ region annealed to the second circular DNA template and a 3′ cleavage fragment; and

amplifying the second circular DNA template by rolling circle amplification primed by a 3′-hydroxyl end of the 5′ region annealed to the second circular DNA template.

In one embodiment, the second circular DNA template comprises a region encoding at least one antisense DNAzyme, optionally a RNA-cleaving DNAzyme. In one embodiment, amplifying the second circular DNA template by rolling circle amplification produces a second amplification product comprising an RNA-cleaving DNAzyme and the RNA-cleaving DNAzyme acts on the RDS nucleic acid molecule on the substrate complex to produce the 5′ cleavage fragment and the 3′ cleavage fragment.

In one embodiment, the 5′ region of the RDS nucleic acid molecule comprises the target sequence and the second circular DNA template comprises a region complimentary to the target sequence.

Optionally, the first circular DNA template and the second circular DNA template may comprise, or consist of, the same DNA sequence. In one embodiment, the first circular DNA template and the second circular DNA template comprise a sequence encoding the same antisense RNA-cleaving DNAzyme.

In one embodiment, the method comprises detecting an increase in the first amplification product and/or second amplification product thereby detecting the presence of the target nucleic acid molecule in the sample.

In one embodiment, there is provided a product or kit comprising one or more of the nucleic acids described herein. In one embodiment, the kit comprises a circular DNA template comprising a region encoding an antisense ribonucleotide-cleaving DNAzyme (RNA-cleaving DNAzyme) and a region complimentary to a target sequence, and a ribonucleotide-containing DNA sequence (RDS) nucleic acid molecule comprising a 5′ region comprising the target sequence and a 3′ region comprising a ribonucleotide that is cleaved by the RNA-cleaving DNAzyme encoded by the circular DNA template. In one embodiment, the 3′ end of the RDS nucleic acid molecule is modified to be resistant to exonuclease activity, optionally by an inverted dT (reverse linkage). Optionally, the kit comprises one or more reagents suitable for rolling circle amplification such as ϕ29DP.

In one embodiment, the 3′ region of the RDS nucleic acid molecule comprises a cleavage site for an exogenous RNA-cleaving DNAzyme, the exogenous RNA-cleaving enzyme is activated by a target analyte, and the kit further comprises the exogenous RNA-cleaving DNAzyme. In one embodiment, the exogenous RNA-cleaving DNAzyme and the RNA-cleaving DNAzyme generated by RCA of the circular template cleave the same RDS on the RDS nucleic acid molecule.

Also provided in the use of a product or kit as described herein as a biosensor for the detection of a target analyte. In one embodiment, there is provided the use of a product or kit in a method for amplifying a target nucleic acid molecule and/or detecting a target analyte as described herein.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows one embodiment of a molecular amplification process and the working principle of DNAzyme feedback amplification (DFA). a) Special DNA assemblies are designed to allow for autonomous, multi-step, cyclic actions from a DNA polymerase (DP) and a DNAzyme (DZ) that can turn a limited number of molecular recognition events into large amounts of DNA amplicons for biosensing applications. b) DP: DNA primer; CDT: circular DNA template; RDS: RNA-containing DNA substrate; RCD: RNA-cleaving DNAzyme; ϕ29DP: ϕ29 DNA polymerase; filled square: a ribonucleotide embedded in a DNA sequence.

FIG. 2 shows the examination of displacement of RDS in Complex II by RP (RCA product of Complex I). a) Schematic illustration of displacement reactions involving RDS in Complex II and RP or MRP (monomeric RP). Successful displacement will result in free RDS and Complex III or Complex IV. Part b) shows electrophoretic mobility shift assay for the reactions shown in part a).

FIG. 3 shows the functionality of MgZ within RP. Part a) shows sequential reactions of cleavage of RDS within Complex I by RP, nucleolytic trimming of cleavage fragment within Complex II and RCA. Note that PNK is required to remove the 2,3-cyclic phosphate. Part b) cleavage reaction with doubly labeled RDS. Part c) shows colorimetric reporting of successful RCA reaction.

FIG. 4 shows DFA reaction for miR-21 detection. Real-time kinetic measurement of 90-minute DFA reactions initiated by miR-21 concentrations varied between 10 nM and 0.1 fM in shown in part a), and in part b) 200-minute DFA reactions initiated by miR-21 concentrations varied between 0.1 pM and 0.1 aM. Part c) shows variation in POI values calculated from 200-minute reactions as a function of miRNA concentration. Part d) shows Specificity of miR-21 detection. The error bars in c) and d) represent standard deviations of three independent experiments.

FIG. 5 shows DFA for E. coli detection. a) Schematic illustration of production of Complex I by EC1 that is activated by E. coli. b) Real-time monitoring of DFA at various E. coli concentrations (cells/mL).

FIG. 6 shows digestion of RDS with ϕ29DP in the presence of CDT, MgZ and PNK. Experimental procedure: A mixture containing 0.5 μM ³²P labelled RDS2 and 1 μM CDT2 in 20 μL of 1×RCA reaction buffer was heated at 90° C. for 1 min and cooled to room temperature for 10 min. MgZ (25 μM) and PNK (0.5 U/μL) were then added. The reaction mixture was incubated at 37° C. for 30 min before heating at 75° C. for 15 min. After cooling at room temperature for 10 min, ϕ29DP (0.5 U/μL) was added and the reaction volume was adjusted to 25 μL using water. After incubation at 37° C. for 30 min, the reaction mixture was analyzed by 10% dPAGE.

FIG. 7 shows detection of miR-21 using a) DFA and b) standard RCA. Experimental procedure: a) DFA reaction: The reaction was performed in 50 μL of 1×RCA reaction buffer containing 300 nM CDT1, 200 nM RDS1, 1 mM dNTPs, 0.2 U/μL PNK, 0.2 U/μL ϕ29DP, 1×SYBR Gold and different amounts of miR-21 targets. These reactions were carried out in a BioRad CFX96 qPCR system set to a constant temperature of 37° C., and the fluorescence intensity was recorded in 1-min intervals. b) Standard RCA: The procedure was similar to that for the DFA reaction except that the reagents were used as follows: 300 nM CDT1, 200 nM DDS, 1 mM dNTPs, 0.2 U/μL PNK, 0.2 U/μL ϕ29DP, 1×SYBR Gold and different amounts of miR-21 targets.

FIG. 8 shows gel electrophoresis analysis of RP produced by the DFA reactions for miR-21 detection. Experimental procedure: RCA reactions were carried out at 37° C. for 200 min in 50 μL of 1×RCA reaction buffer containing 300 nM CDT1, 200 nM RDS1, 1 mM dNTPs, 0.2 U/μL PNK, 0.2 U/μL ϕ29DP, and different amounts of miR-21 targets. The resultant mixture was heated at 90° C. for 10 min. 1 μL of the reaction mixture was then mixed with 5 μL of DT1 (100 μM), heated at 90° C. for 5 min and cooled at room temperature for 10 min. This was followed by the addition of 2 μL of 10× Fast digestion buffer and 3 μL of EcoRV. The reaction mixture was then incubated at 37° C. for 12 h. This procedure was designed to convert long RP products into monomeric units (77 nt) to facilitate gel-based DNA concentration analysis. After combining with 2 μL of 1 μM DC1 (62 nt, a reference DNA for gel analysis), the digested RP was analyzed by 10% dPAGE gel and stained with 1×SYBR Gold. Fluorescence ratio (FR) was then calculated for DFA reactions at each miR-21 concentration.

FIG. 9 shows cleavage of RDS by EC1. Experimental procedure: The cleavage reactions were carried out at 37° C. for 30 min in 20 μL of 1×RB containing 5′-′²P labelled RDS3 (0.5 μM), EC1 (25 μM) or EC1M (inactive mutant EC1; 25 μM), and 5 μL of E. coli CIM (prepared from 1 mL of E. coli cells at a concentration of 10⁶ cells/mL). The reaction mixture was then analyzed by 10% dPAGE.

FIG. 10 shows agarose gel analysis of the DFA reaction for E. coli detection. Experimental procedure: RCA reactions were carried out at 37° C. for 60 min in 20 μL of 1×RB containing indicated components of CDT1 (300 nM), RDS3 (200 nM), EC1 (10 μM), 5 μL of E. coli CIM (prepared from 1 mL of E. coli cells at a concentration of 10⁶ cells/mL) and 0.5 U/μL PNK, followed by addition of 5 μL of 10×RCA buffer, 0.2 U/μL ϕ29DP and 1 mM dNTPs, and incubation at 37° C. for 30 min before agarose gel analysis (0.6%).

FIG. 11 shows gel electrophoresis analysis of RP produced by the DFA reactions for E. coli detection. Experimental procedure: RCA reactions were carried out at 37° C. for 60 min in 20 μL of 1×RB buffer containing 300 nM CDT1, 200 nM RDS3, 10 μM EC1, 5 μL of E. coli CIM (prepared from 1 mL of E. coli at indicated concentrations) and 0.5 U/μL PNK, followed by addition of 5 μL of 10×RCA buffer, 0.2 U/μL ϕ29DP and 1 mM dNTPs, and incubation at 37° C. for 30 min. 1 μL of the reaction mixture was mixed with 5 μL of DT1 (100 μM), heated at 90° C. for 5 min and cooled at room temperature for 10 min. This was followed by the addition of 2 μL of 10× Fast digestion buffer and 3 μL of EcoRV. The reaction mixture was then incubated at 37° C. for 24 h. This procedure was designed to convert long RP molecules into MRP (77 nt) to facilitate gel-based DNA concentration analysis. After combining with 2 μL of 1 μM DC1 (a DNA control for gel analysis), the digested RP was analyzed by 10% dPAGE gel and stained with 1×SYBR Gold. Fluorescence ratio (FR) was then calculated for each DFA reaction at each E. coli concentration.

FIG. 12 shows specificity of the DFA reaction for E. coli detection. The gram-negative bacteria used were Achromobacter xylosoxidans (AX), Yersinia ruckeri (YR) and Hafnia alvei (HA). The gram-positive bacteria used were Pediococcus acidilactici (PA) and Bacillus subtilis (BS). Experimental procedure: Experiments were carried out at 37° C. for 60 min in 20 μL of 1×RCA buffer containing 300 nM CDT1, 200 nM RDS3, 10 μM EC1, 5 μL of bacterial CIM (prepared from 1 mL of a specific bacterium at the concentration of 10⁶ cells/mL) and 0.5 U/μL PNK, followed by the addition of 5 μL of 10×RCA buffer, 0.2 U/μL ϕ29DP, 1 mM dNTPs and 1×SYBR Gold. The fluorescence intensity was recorded in 1-min intervals using a BioRad CFX96 qPCR system.

### DETAILED DESCRIPTION

Described herein are methods for the amplification of a signal generated by rolling circle amplification (RCA) in response to a target analyte. RCA of a circular template produces an RNA-cleaving DNAzyme which acts on a substrate complex with a ribonucleotide-containing DNA sequence (RDS) to generate additional circular DNA templates for RCA, thereby providing feedback amplification of the initial RCA triggered by the target analyte. Also described are methods for the detection of a target analyte as well as products and kits comprising a circular DNA template and/or an RDS. In one embodiment, the products and kits are useful for the detection of a target analyte in a biological and/or environmental sample. The embodiments described herein may also be used for amplifying and/or detecting a target analyte in a biosensor, optionally a hand held biosensor or other portable device. Methods that employ DNAzyme feedback amplification as described herein are highly sensitive and can readily be adapted for the detection of different target analytes including, but not limited to, nucleic acid molecules such as mRNA or miRNA or ssRNA or ssDNA viruses.

### I. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “target analyte” as used herein means any agent, including, but not limited to, nucleic acids, small inorganic and organic molecules, metal ions, hormonal growth factors, biomolecules, toxins, biopolymers (such as carbohydrates, lipids, peptides and proteins), cells, tissues and microorganisms (including bacteria and viruses), for which one would like to sense or detect. In an embodiment, the target analyte is either from a natural source or is synthetic. In one embodiment, the target analyte may be a single compound or a class of compounds, such as a class of compounds that share structural or functional features. In one embodiment, the target analyte is a target nucleic acid molecule comprising a target sequence. In some embodiments, the target sequence is unique to the target analyte or may be associated with a class of target analytes. In one embodiment, the target analyte is a messenger RNA (mRNA) or micro RNA (miRNA). In one embodiment, the target analyte is a nucleic acid molecule from a microbial pathogen, optionally a bacteria, single stranded RNA virus or a single stranded DNA virus. In one embodiment, the target analyte is a protein optionally a biomarker associated with a microbial pathogen or disease. In one embodiment, the target analyte may be a fragment of a larger analyte such as a biomolecule. For example, in one embodiment, the target analyte is target nucleic acid molecule that is the product of digesting a nucleic acid molecule with a restriction enzyme.

In one embodiment, the target analyte directly or indirectly triggers RCA of a circular DNA template as described herein. For example, the target analyte may be a target nucleic acid molecule that binds to a complimentary sequence on a circular DNA template and a 3′-hydroxyl end of the target nucleic acid molecule serves as a primer for RCA. In another embodiment, the target analyte binds to a recognition moiety and that binding event directly or indirectly triggers RCA of a circular DNA template as described herein.

The term “recognition moiety” as used herein refers to an agent that is able to recognize the presence of an analyte. Recognition moieties, include without limitation, aptamers, structure-switching aptamers, reporter aptamers, DNAzymes, antibodies, and nucleic acid probes.

The term “aptamer” as used herein refers to short, chemically synthesized, single stranded (ss) RNA or DNA oligonucleotides which fold into specific three-dimensional (3D) structures that bind to a specific analyte with dissociation constants, for example, in the pico- to nano-molar range.

The term “structure-switching nucleic acid aptamers” or “reporter aptamers” as used herein refers to aptamers that function by switching structures from a DNA/DNA or RNA/RNA complex to a DNA/analyte or RNA/analyte complex. For example, in one embodiment a DNAzyme comprising a structure-switching nucleic acid aptamer may form a complex with a target analyte and switch structures to a catalytically active form of the DNAzyme that directly or indirectly triggers rolling circle amplification of a circular DNA template.

The term “concatemeric nucleic acid molecules” or “concatemer” as used herein refers to a long continuous DNA or RNA molecule that contains multiple copies of the same DNA or RNA sequences linked in a tandem series. In one embodiment, the amplification products of the circular DNA constructs described herein are concatemers comprising a plurality of the same sequence encoding a DNAzyme and a sequence complimentary to a target sequence.

The term “rolling circle amplification” as used herein refers to a unidirectional nucleic acid replication that can rapidly synthesize multiple copies of circular molecules of DNA or RNA. In an embodiment, rolling circle amplification is an isothermal enzymatic process where a short DNA or RNA primer is amplified to form a long single stranded DNA or RNA using a circular DNA template and an appropriate DNA or RNA polymerase. The product of this process is a concatemer that may contain ten to thousands of tandem repeats that are complementary to the circular template.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer is sufficiently long to prime the synthesis of the desired extension product in the presence of the inducing agent. The exact length of the primer will depend upon factors, including temperature, sequences of the primer and the methods used. In one embodiment, a primer is a nucleic acid molecule that has a free 3′-hydroxyl end. It can be DNA, RNA, or a chimeric DNA/RNA sequence. In some embodiments, a target nucleic acid molecule that binds to a complementary region on a circular DNA template acts as a primer for rolling circle amplification. In other embodiments, the 3′ end of a cleavage fragment annealed to a circular DNA template, wherein any unpaired nucleotides at the 3′ end have been removed, acts as a primer for rolling circle amplification.

In one embodiment, complimentary regions of nucleic acid molecules as described herein may anneal or hybridize to one another according to established principles of base pairing known in the art. The term “probe” refers to a nucleic acid sequence that will hybridize to a nucleic acid target sequence. The length of probe depends on the hybridization conditions and the sequences of the probe and nucleic acid target sequence. In one embodiment, the probe is 8-100, 8-200 or 8-500 nucleotides in length, such as 8-10, 11-15, 16-20, 21-25, 26-50, 51-75, 76-100, 101-150 or 151-200 nucleotides in length or at least 200, 250, 400, 500 or more nucleotides in length. In other embodiments, 10, 15, 20 or 25 nucleotides provide a lower end for the aforementioned nucleotide ranges. In one embodiment, a target nucleic acid molecule as described herein may act as a probe and anneal to a complimentary sequence on a circular template. In another embodiment, the 5′ end of a RDS nucleic acid molecule as described herein may act as a probe and anneal to a complimentary sequence on a circular template. Optionally, a probe may act as a primer to initiate rolling circle amplification of a circular template.

It will be appreciated that in some embodiments, a primer or probe may contain non-complementary sequences provided that a sufficient amount of the primer or probe contains a sequence which is complementary to a region of the circular template disclosed herein, to allow hybridization of the primer or probe to the circular template. In one embodiment, the primer or probe binds to the circular template under at least moderately stringent hybridization conditions so as to prevent non-specific amplification of the circular DNA template.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In some embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm—5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001. In one embodiment, the target nucleic acid molecule binds to a circular DNA template under at least moderately stringent hybridization conditions. In one embodiment, the RDS nucleic acid molecule binds to a circular DNA template under at least moderately stringent hybridization conditions.

The term “circular template” as used herein refers to a nucleic acid sequence of at least 20 nucleotides that is ligated to form a circular nucleic acid molecule that can serve as a template for rolling circle amplification. In one embodiment, the circular template comprises DNA. In one embodiment, the circular template encodes for one or more antisense DNAzymes such that amplification of the circular template by rolling circle amplification produces an amplification product that is the complement of the circular DNA template comprising the DNAzyme sequence.

As used herein, “deoxyribozyme” or “DNAzyme” refers to a nucleic acid molecule comprising DNA that is capable of performing a specific chemical reaction. In some embodiments, a DNAzyme may comprise, or be complexed with or conjugated to, an DNA aptamer that binds selectively to a target analyte. Methods for generating DNA aptamers and/or DNAzymes are known in the art and, for example, can be identified from random DNA pools via in vitro selection as described in [7]. In one embodiment, the DNAzyme is capable of acting on a substrate to generate a detectable signal. For example, PW17 is a DNAzyme capable of inducing a color change by oxidizing a chromogenic substrate.

In one embodiment, the DNAzyme is capable of cleaving a DNA molecule comprising a ribonucleotide i.e. “a ribonucleotide-cleaving DNAzyme” “RNA-cleaving DNAzyme”, or “RCDs”. Examples of RCDs known in the art include MgZ (described in [12]), as well as RCDs that are useful for detecting target bacteria such E. coli (see EC1 described in [18]) or Clostridium difficile (described in [11g]), or biomarkers for cancer cells (described in [26]). Methods suitable for generating RCDs for specific ribonucleotide containing target sequences are also described in [11g], [18] and [26] (all of which are hereby incorporated by reference) including processes such as the systematic evolution of ligands by exponential enrichment (SELEX). Methods such as SELEX known in the art may also be used to generate exogenous RNA-cleaving DNAzymes that are activated by a target analyte to cleave a RDS sequence. As set out above, the target analyte may be, without limitation, a biomolecule such as a protein, nucleic acid, carbohydrate or lipid. In one embodiment the target analyte is a biomarker associated with a microbial pathogen or disease.

As used herein, “ribonucleotide-containing DNA sequence”, “RNA-containing DNA sequence”, or “RDS” refers to a DNA sequence comprising one or more ribonucleotides that is recognized and cleaved by a RNA-cleaving DNAzyme. In one embodiment, the RDS comprises a single ribonucleotide and the remaining nucleotides in the sequence are deoxyribonucleotides. In one embodiment, the RDS comprises two DNA sequences linked by a single ribonucleotide, optionally wherein one or both DNA sequences is at least 2, 3, 4, 5, 6, 7, 8, 9, or 10 nucleotides in length, optionally between 5 and 20 nucleotides in length. In one embodiment, the ribonucleotide is a nucleotide containing ribose as its pentose component. In one embodiment, the nucleotide comprises a base selected from adenine (A), guanine (G), cytosine (C), or uracil (U). For example, the RDS may contain Adenine ribonucleotide (rA), Guanine ribonucleotide (rG), Cytosine ribonucleotide (rC), or Uracil ribonucleotide (rU). Examples of RDS nucleic acid molecules include but are not limited to RDS1 (SEQ ID NO: 5), RDS2 (SEQ ID NO: 6), RDS3 (SEQ ID NO: 7) and DDS (SEQ ID NO: 8) found in Table 1. Optionally, the RDS nucleic acid molecule comprises a detectable marker. For example, in one embodiment the RDS nucleic acid molecule comprises fluorophore (F) and quencher (Q) moieties on either side of the ribonucleotide, such that cleavage by an RNA-cleaving DNAzyme dissociates the quencher and fluorophore resulting in a detectable signal.

In one embodiment, a RDS nucleic acid molecule described herein comprises a 5′ region and a 3′ region comprising the ribonucleotide sequence that is recognized by the RNA-cleaving DNAzyme. In one embodiment, the 5′ region comprises a sequence that serves as a probe and/or primer that binds to a complimentary sequence on a circular DNA template.

### II. Methods, Products, Kits and Associated Uses

In one embodiment, there is provided a method for detecting a target analyte in a sample comprising:

combining the sample with a first circular DNA template comprising a region encoding an antisense ribonucleotide-cleaving DNAzyme (RNA-cleaving DNAzyme), wherein the first circular DNA template is amplified by rolling circle amplification in the presence of the target analyte to produce a first amplification product comprising the RNA-cleaving DNAzyme;

contacting the first amplification product comprising the RNA-cleaving DNAzyme and a substrate complex comprising a ribonucleotide-containing DNA sequence (RDS) nucleic acid molecule annealed to a second circular DNA template, the second circular DNA template comprising a region encoding an antisense DNAzyme and a region complimentary to the 5′ end of the RDS nucleic acid molecule, wherein the RNA-cleaving DNAzyme acts on the substrate complex to produce a 5′ cleavage fragment comprising a 5′ region annealed to the second circular DNA template and a 3′ cleavage fragment;

amplifying the second circular DNA template by rolling circle amplification primed by a 3′-hydroxyl end of the 5′ region annealed to the second circular DNA template to produce a second amplification product comprising the DNAzyme; and

detecting an increase in the first amplification product and/or second amplification product thereby detecting the presence of the target analyte in the sample.

Various mechanisms may be used to initiate RCA of the first circular DNA template in response to a target analyte. For example, in one embodiment, the target analyte is a target nucleic acid molecule that binds to the first circular DNA template and RCA of the first circular DNA template is primed by a 3′-hydroxyl end of the target nucleic acid molecule that binds to the first circular DNA template. In another embodiment, the target analyte activates a RNA-cleaving DNAzyme, such as EC1, that binds to an RDS sequence annealed to the first circular DNA template to produce a 5′ cleavage product comprising a 5′ region annealed to the first circular DNA template and RCA of the first circular DNA template is primed by a 3′-hydroxyl end of the 5′ region annealed to the first circular DNA template.

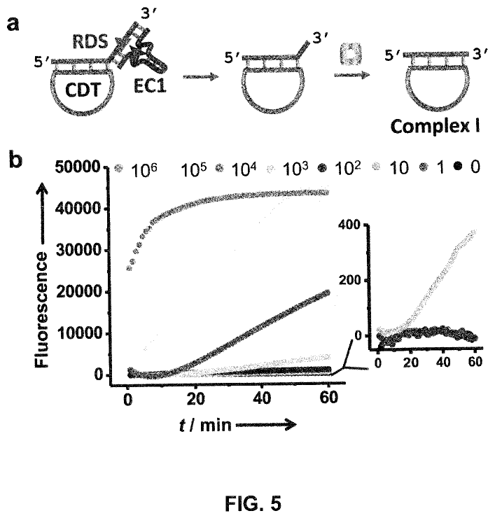

In one embodiment, the target analyte activates an exogenous RNA-cleaving DNAzyme that binds to a nucleic acid molecule annealed to the first circular DNA template comprising one or more RDS sequences to produce a 5′ cleavage product comprising a 5′ region annealed to the first circular DNA template, wherein rolling circle amplification of the first circular DNA template is primed by a 3′-hydroxyl end of the 5′ region annealed to the first circular DNA template. One embodiment wherein an exogenous RNA-cleaving DNAzyme is used to activate RCA is shown in FIG. 5. In one embodiment, the nucleic acid molecule annealed to the first circular DNA template comprises a first RDS sequence that is cleaved by the exogenous RNA-cleaving DNAzyme and a second RDS sequence that is cleaved by an RNA-cleaving DNAzyme encoded by the second circular template. In one embodiment, the exogenous RNA-cleaving DNAzyme and the RNA-cleaving DNAzyme generated by RCA of the circular template cleave the same RDS on the RDS nucleic acid molecule.

In another embodiment, the target analyte binds to a recognition moiety that directly or indirectly triggers rolling circle amplification of the first circular DNA template.

Also provided is a method of amplifying a target sequence of a target nucleic acid molecule. In one embodiment, the method comprises:

combining the sample and a first circular DNA template comprising a region encoding an antisense ribonucleotide-cleaving DNAzyme (RNA-cleaving DNAzyme) and a region complimentary to the target sequence such that a 3′-hydroxyl end of the target nucleic acid molecule anneals to the region complimentary to the target sequence on the circular DNA template;

amplifying the first circular DNA template by rolling circle amplification primed by the 3′-hydroxyl end of the target nucleic acid molecule to produce a first amplification product comprising a RNA-cleaving DNAzyme, wherein the RNA-cleaving DNAzyme acts on a substrate complex comprising a ribonucleotide-containing DNA sequence (RDS) nucleic acid molecule annealed to a second circular DNA template to produce a 5′ cleavage fragment comprising a 5′ region annealed to the second circular DNA template and a 3′ cleavage fragment; and

amplifying the second circular DNA template by rolling circle amplification primed by a 3′-hydroxyl end of the 5′ region annealed to the second circular DNA template.

Optionally, the first and/or second circular DNA templates encode an antisense sequence for at least one DNAzyme, optionally a RNA-cleaving enzyme such as MgZ or EC1. In one embodiment, the first and/or second circular DNA templates encode a plurality of antisense sequences for the same DNAzyme.

In one embodiment, the DNAzyme encoded by the second amplification product acts on a substrate to produce a detectable signal. For example, the DNAzyme PW17 produces a colorimetric signal by acting on the chromogenic substrate ABTS.

In one embodiment, the DNAzyme encoded by the second amplification product is an RNA-cleaving DNAzyme, optionally the same RNA-cleaving DNAzyme that is encoded by the first amplification product. In one embodiment, the RNA-cleaving DNAzyme on the second amplification product acts on the RDS nucleic acid molecule on the substrate complex to produce the 5′ cleavage fragment and the 3′ cleavage fragment. In one embodiment, this generates DNAzyme feedback amplification generating more input complexes for rolling circle amplification.

In one embodiment, the 5′ region of the RDS nucleic acid molecule comprises the target sequence and the second circular DNA template comprises a region complimentary to the target sequence. In one embodiment, the first circular DNA template and the second circular DNA template comprise or consist of the same DNA sequence.

In one embodiment, the substrate complex is formed prior to combining the sample and a first circular DNA template. In one embodiment, method comprises combining the RDS nucleic acid molecule and a stoichiometric excess of the first circular DNA template to form a mixture comprising the first circular DNA template and the substrate complex. In one embodiment, the step of combining the sample and the first circular DNA template comprises combining the sample and the mixture. In one embodiment, the stoichiometric excess of the first circular DNA template relative to the RDS nucleic acid molecule is at least 3:2.

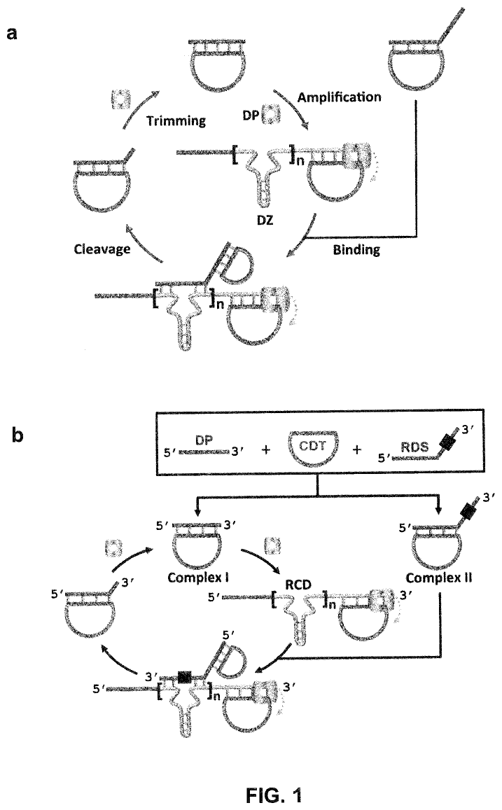

As shown in FIGS. 1a and 1b, prior to serving as a primer for rolling circle amplification of a circular template, any unpaired nucleotides from the 5′ cleavage fragment should be removed to form the 3′-hydroxyl end of the 5′ region annealed to the second circular DNA template 5′ region which can then act as a primer for RCA. Accordingly, in one embodiment the method comprises contacting the 5′-cleavage fragment with an enzyme to trim or remove unpaired nucleotides. In one embodiment, an enzyme with 3′-5′ exonuclease activity is used to remove any unpaired nucleotides, optionally wherein the enzyme is phi 29 DNA polymerase (ϕ29DP). In one embodiment, the method comprises contacting the 5′-cleavage fragment with an enzyme with exonuclease activity in the presence of polynucleotide kinase (PNK) which is used to remove 2,3-cyclic phosphate.

The 3′ end of the RDS nucleic acid molecule shown in FIG. 1b as Complex II may be modified to prevent degradation of the single stranded 3′ region. This prevents the 3′ end from being degraded such that rolling circle amplification of the circular DNA template could be initiated prior to cleavage by the RNA-cleaving DNAzyme. For example, in one embodiment, the 3′ end of the RDS nucleic acid molecule comprises a 3′ Inverted dT leading to a 3′-3′ linkage which inhibits both degradation by 3′ exonucleases and extension by DNA polymerases. A number of different techniques may be used for modifying the 3′ end of the RDS nucleic acid molecule to prevent degradation. For example, phosphorothioate (PS) bonds, 2′-O-Methyl (2′OMe), 2′ Fluoro bases, an phosphoramidite C3 Spacer and/or other modifications such as inverted dT may be used alone or in combination, to confer resistance to exonuclease activity.

Conditions and reagents suitable for rolling circle amplification (RCA) of a circular template are known in the art. For example, RCA occurs in the presence of a polymerase that possesses both strand displacement ability and high processivity in the presence of template, primer and nucleotides. In an embodiment, RCA conditions comprise temperatures of from about 20° C. to about 40° C., optionally at room temperature or at an elevated temperature such as about 30° C. or about 37° C. In an embodiment, RCA conditions comprise a reaction time sufficient for the generation of detectable amounts of amplicons, optionally at least 15 minutes, 30 minutes, 60 minutes, 100 minutes, 120 minutes, 160 minutes or 200 minutes. In one embodiment, RCA conditions comprise performing the reaction in a buffer, such as a commercially available RCA buffer. In an embodiment, ϕ29-, Bst-, or Vent exo-DNA polymerase is used for RCA of the first circular DNA template and/or second circular DNA template. In an embodiment, the rolling circle amplification conditions comprise the presence of ϕ29DP.

In one embodiment, RCA of the circular DNA template is an isothermal process. In one embodiment, the steps of combining the sample with the first circular DNA template, contacting the first amplification product and the substrate complex, and amplifying the second circular DNA template are done at the same temperature. In one embodiment, one or more steps of combining the sample with the first circular DNA template, contacting the first amplification product and the substrate complex, and amplifying the second circular DNA template are done at a temperature between about 20° C. and 40° C., optionally at room temperature, or at an elevated temperature such as around 30° C. or 37° C. In one embodiment, one or more steps of the method are done at an ambient temperature, optionally between about 15° C. and 30° C., or between about 18° C. and 25° C. In one embodiment, the method is performed at an ambient temperature without heating or cooling the sample.

In one embodiment, the methods described herein can advantageously be performed in a single reaction vessel. For example, in one embodiment, the combining the sample with the first circular DNA template, contacting the first amplification product and the substrate complex, and amplifying the second circular DNA template are done in the same reaction vessel. Optionally, the reaction vessel may have a pre-determined quantity of reagents suitable for performing a method as described herein. In one embodiment, the reagents are freeze-dried, optionally on a substrate and are activated by addition of the sample in solution. In one embodiment, the methods and/or reagents described herein are incorporated on a substrate, optionally a paper based substrate or other solid support, suitably for use in point-of-case diagnostic biosensors. In one embodiment, the methods and/or reagents described herein are encapsulated in a stabilizing matrix. Embodiments, wherein the RCA reactions are incorporate on a substrate and/or encapsulated in a stabilizing matrix, as described in WO2017096491 (hereby incorporated by reference).

In one embodiment, the target analyte is a target nucleic acid molecule. Alternatively, the target analyte may be any substance that selectively triggers RCA of the first circular DNA template. Methods for generating a RNA-cleaving DNAzyme that is activated by the presence of a target analyte, which can then go on to cleave a target RDS nucleic acid molecule and trigger RCA may be generated using methods known in the art such as SELEX process and/or set out in references [11g], [18] and [26]. IN one embodiment, the target analyte is biomarker, such as a protein, carbohydrate or lipid. In one embodiment, the target nucleic acid molecule is a single stranded DNA molecule, optionally a ssDNA virus. In another embodiment, the target nucleic acid molecule is a single stranded RNA molecule, optionally a microRNA (miRNA) molecule, or an mRNA molecule, or a ssRNA virus. In one embodiment, the target analyte, optionally a target nucleic acid molecule, is associated with a disease or microbe, optionally a microbial pathogen. In one embodiment, the microbe is a virus or bacteria. Associations between target analytes and specific diseases and/or microbes are well known in the art. For example, as set out in the Examples certain miRNAs such as miR-21 are biomarkers for cancer diagnosis and prognosis.

In one embodiment the sample is any sample for which information regarding the presence or absence of a target analyte is desired. For example, the sample may be a biological sample from a subject such as a blood sample, tissue sample, urine sample, stool sample or cerebrospinal fluid. In one embodiment, the sample is an environmental sample, such as a sample associated with a specific location or place. In one embodiment, the environmental sample is a water sample or air sample. In one embodiment, the environmental sample is a sample obtained by swabbing or contacting a surface or material.

In one embodiment, the sample is treated prior combining the sample and the first circular DNA template. In one embodiment, the sample is treated to remove any components that may interfere with methods described herein. For example, the sample may be heated to denature any proteins and/or render nucleic acid molecules single stranded. Optionally, the sample may be treated with a restriction enzyme in order to generate a target nucleic acid molecule.

In one embodiment, the methods described herein include detecting an increase in a level of first and/or second amplification products generated by RCA. Methods for detection of products of rolling circle amplification are known in the art, such as but not limited to colorimetric, electrochemical and/or spectroscopic methods. Detecting an increase in a level of the first and/or second amplification products includes qualitative and/or quantitative detection. In one embodiment, detecting an increase in a level of first and/or second amplification products comprises detecting a signal generated by the activity of a DNAzyme encoded by the first or second amplification products. In another embodiment, detecting an increase in a level of first and/or second amplification products comprises detecting an increase of polymeric nucleic acids. For example, various dyes that selectively bind to polymeric nucleic acids may be used. In one embodiment, the methods described herein include contacting the sample with a fluorescent dye that binds to DNA. These include, but are not limited to, ethidium bromide or cyanine-based dyes such as SYBR™ Green or SYBR™ Gold.

Circular DNA templates as described herein can readily be generated using methods known in the art. For example, in one embodiment a circular DNA template is generated by selecting a linear sequence comprising a region complementary to the target sequence and one or more sequences encoding one or more antisense RNA-cleaving DNAzymes, followed by circularizing the sequence to form the circular DNA template. In an embodiment, the circularization is performed using DNA ligase, such as T4 DNA ligase.

In another aspect, there are provided nucleic acid molecules and/or associated complexes as described herein that are useful for DNAzyme feedback amplification. In one embodiment, there is provided a kit comprising a circular template as disclosed herein and/or an RDS nucleic acid molecule, and optionally one or more reagents necessary for carrying out rolling circle amplification, such as a suitable DNA polymerase, NTPs, reaction buffer, or instructions for use. In one embodiment, the DNA polymerase is phi29 DNA polymerase.

In one embodiment, the kit comprises:

a circular DNA template comprising a region encoding an antisense ribonucleotide-cleaving DNAzyme (RNA-cleaving DNAzyme) and a region complimentary to a target sequence; and

a ribonucleotide-containing DNA sequence (RDS) nucleic acid molecule comprising a 5′ region comprising the target sequence and a 3′ region comprising a ribonucleotide that is cleaved by the RNA-cleaving DNAzyme encoded by the circular DNA template.

In one embodiment, the circular DNA template and the RDS nucleic acid molecule are in the same container or alternatively in separate containers. In one embodiment, the circular DNA template and the RDS nucleic acid molecule are in the same container and the stoichiometric ratio of the circular DNA template and the RDS nucleic acid molecule is at least 3:2.

In one embodiment, the RNA-cleaving DNAzyme is MgZ. In one embodiment, the circular DNA template encodes for the antisense of a RNA-cleaving DNAzyme sequence provided in Table 1, such as MgZ (SEQ ID NO: 9).

In one embodiment, the 3′ end of the RDS nucleic acid molecule is modified to prevent exonuclease degradation. For example, in one embodiment the 3′ end of the RDS nucleic acid molecule comprises a 3′ Inverted dT leading to a 3′-3′ linkage which inhibits both degradation by 3′ exonucleases and extension by DNA polymerases. Alternatively or in addition, the 3′ end of the RDS nucleic acid molecule may be modified using Phosphorothioate (PS) bonds, 2′-O-Methyl (2′OMe), 2′ Fluoro bases, an phosphoramidite C3 Spacer and/or other modifications to confer resistance to exonuclease activity.

In one embodiment, the kit comprises a RDS nucleic acid molecule that is recognized and cleaved by two or more different RNA-cleaving DNAzymes. For example, in one embodiment the RDA nucleic acid molecule has a RDS that is cleaved by an exogenous RNA-cleaving DNAzyme that is activated by the presence of a target molecule and an RDS sequence that is cleaved by a RNA-cleaving DNAzyme generated by RCA of a circular DNA template.

In one embodiment, the kit further comprises an exogenous RNA-cleaving DNAzyme that is activated by a target analyte. Examples of such a RNA-cleaving DNAzyme include EC1 as well as the DNAzymes described in [11g] or [26]. In one embodiment, the exogenous RNA-cleaving DNAzyme comprises an aptamer sequence that binds to a target analyte causing a structural shift that activates the RNA-cleaving DNAzyme.

Also provided is the use of a kit as described herein in a biosensor for the detection of a target analyte. Also provided is the use of a kit as described herein for performing a method for detecting a target analyte or amplifying a target nucleic acid molecule as described herein.

In one embodiment, there is provided a biosensor comprising:
i) a RNA-cleaving DNAzyme;
ii) a circular DNA template, encoding the antisense sequence of the RNA-cleaving DNAzyme;
iii) a nucleic acid primer that is complimentary to part of the circular DNA template; and
iv) a DNA strand containing an internal ribonucleotide linkage wherein the 5′ end encodes the same nucleic acid sequence of the nucleic acid primer that is complimentary to the circular DNA template,
wherein binding of the nucleic acid prime to the circular DNA template leads to generation of RNA-cleaving DNAzymes by rolling circle amplification and cleavage of the DNA strands containing an internal ribonucleotide linkage leading to additional DNA assemblies for rolling circle amplification that can be detected.

In one embodiment, the nucleic acid primer is a target analyte comprising a target nucleic acid molecule. In one embodiment, the nucleic acid primer is a miRNA molecule. In one embodiment, the RNA-cleaving DNAzyme is activated by the presence of a target microorganism.

In one embodiment, there is provided a method for detecting a target analyte such as miRNA in a sample. In one embodiment, the method comprises exposing the sample to a biosensor, wherein the biosensor is activated by (i) a target analyte, such as miRNA, binding to a complimentary sequence of a circular DNA template, (ii) cleavage of a DNA strand containing an internal ribonucleotide linkage and (iii) generation of single stranded DNA by rolling circle amplification that can be detected.

Also provided is a method for detecting a microorganism in a sample. In one embodiment, the method comprises exposing the sample to a biosensor, wherein the biosensor is activated by (i) interaction of a RNA-cleaving DNAzyme with a microorganism target, (ii) cleavage of a DNA strand containing an internal ribonucleotide linkage and (iii) generation of single stranded DNA by rolling circle amplification that can be detected.

### EXAMPLES

The following non-limiting examples are illustrative of the present application:

### Example 1. Working Principle and Functional Verification of DFA

As shown in FIG. 1(b), the DFA system is made of a DNA primer (DP), a circular DNA template (CDT), and an RNA-containing DNA sequence (RDS) acting as the substrate of an RCD (Table S1 in the Supporting Information lists the sequences of all DNA molecules). CDT and RDS are designed to have two important features: (1) CDT contains the antisense sequence of the RCD, and (2) the 5′ portion of the RDS sequence is identical to that of DR Mixing these three DNA molecules will create two DNA complexes: a DP/CDT hybrid (Complex I) and an RDS/CDT hybrid (Complex II). These complexes enable the following chain of reactions: (a) RCA on Complex I is carried out by ϕ29DP, producing long RCA products containing repetitive RCD units; (b) binding occurs between the resultant RCD elements and the 3′ portion of RDS in Complex II; (c) cleavage of the RNA unit of the RDS by the RCDs occurs, producing the hybrid of CDT with the 5′ cleavage fragment; (d) trimming of unpaired nucleotides of the cleavage fragment by ϕ29DP produces more Complex I, feeding back into the RCA process. This chain of reactions is expected to continue autonomously, resulting in exponential DNA amplification. In theory, any RCD can be used to carry out DFA. For this study, MgZ, an RCD previously published by our group^{[12]} was used.

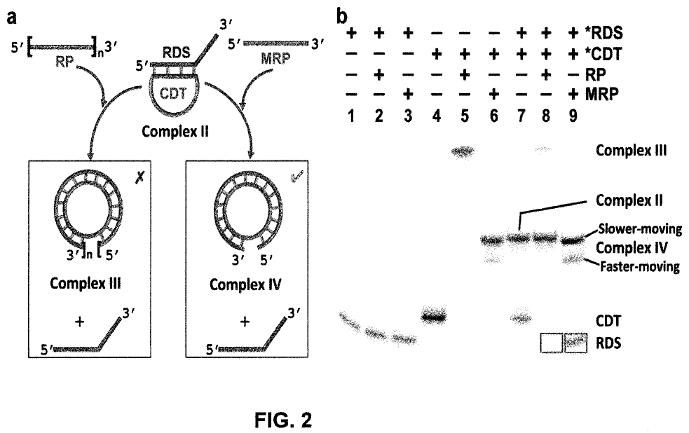

Three conditions should preferably be met for a successful DFA process. First, the RCA product (named RP) should not cause the dissociation of RDS from Complex II. Because RP contains tandem copies of the complement to CDT, it has the potential to displace RDS from Complex II and form an RP/CDT hybrid (Complex III; left pathway in FIG. 2a). To determine if this could happen, an electrophoretic mobility shift assay was carried out using non-denaturing polyacrylamide gels and radioactive RDS, radioactive CDT, or both (FIG. 2b). As a control, monomeric RP (MRP) was also tested, which was expected to form Complex IV with CDT (right pathway, FIG. 2a). Lanes 1-7 represent various controls to show: 1) both RP (lane 2) and MRP (lane 3) do not bind RDS; 2) RP (lane 5) and MRP (lane 6) do bind CDT; 3) RDS and CDT do form Complex II (lane 7; note that CDT was used in excess over RDS in the experiment). It is noteworthy that two forms of Complex IV were observed (lane 6), a faster-moving species (minor product) and a slower-moving species (major product). Based on the observation that the slower-moving species closely matched the gel mobility of Complex II (which contains both ds and ss DNA elements), the faster-moving variant is speculated to be the full circular DNA duplex and the slower-moving one is a CDT/MRP hybrid containing both ds and ss DNA elements. Importantly, RDS were not observed when RP was added into the RDS/CDT mixture (red box, lane 8). In contrast, RDS was seen when MRP was added (green box, lane 9). These results show that although MRP can displace RDS from Complex II, displacement does not happen with RP. The lack of displacement is likely due to the fact that tandemly repetitive sequence units within RP do not have free ends to hybridize with CDT efficiently.

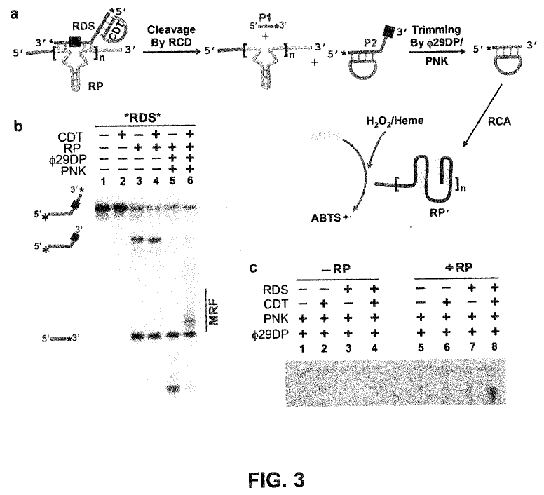

Second, functional DFA also requires that the RCD elements within RP are catalytically active. An experiment was carried out to examine the cleavage activity of RP using doubly labeled radioactive RDS (to track both cleavage fragments). As shown in FIG. 3a, both free RDS (lane 3) and RDS in Complex II (lane 4) can indeed be cleaved by RP.

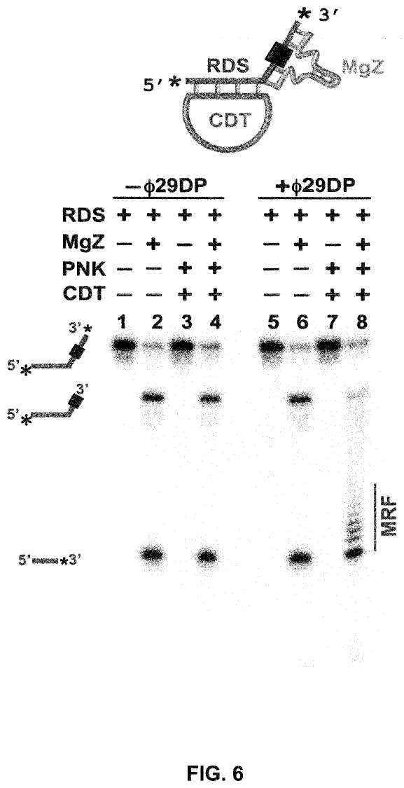

The final requirement is that any overhanging 3′-nucleotides of the 5′-cleavage fragment in Complex II are removed by ϕ29DP or by another reagent. This was confirmed experimentally (FIG. 3b): while the 5′ cleavage fragment alone was fully digested by ϕ29DP (lane 5), only unpaired nucleotides were trimmed when complexed with CDT, as revealed by the appearance of mid-range fragments (MRF; lane 6). These observations are identical to the ones obtained with monomeric MgZ and Complex II (FIG. 6).

Next the MRF-primed RCA reaction was tested. CDT was redesigned so that RCA would generate PW17, a peroxidase-mimicking DNAzyme capable of inducing a color change by oxidizing the chromogenic substrate ABTS (2,2′-azino-bis(3-ethylbenzothiazoline-6-sulfonate)) in the presence of hemin and hydrogen peroxide.^{[13]} For this experiment, the MgZ-containing RP was produced using the original CDT and then mixed with RDS complexed with the altered CDT in the presence of ϕ29DP and PNK. The RP was expected to cleave RDS and generate the P2/CDT complex that could be trimmed by PNK/ϕ29DP (FIG. 3a). The trimmed complex should undergo RCA, generating a PW17-containing RCA product, RP′, that can oxidize ABTS to produce a color change (FIG. 3a). This expectation was confirmed experimentally (FIG. 3b; lane 8, and other lanes represent various controls).

### Methods and Experimental Details

### Materials

The sequences of all DNA and RNA oligonucleotides are provided in Table 1. They were purchased from Integrated DNA Technologies (IDT) and purified by 10% denaturing (8 M urea) polyacrylamide gel electrophoresis (dPAGE) or high performance liquid chromatograph (HPLC). T4 polynucleotide kinase (PNK), T4 DNA ligase and ϕ29 DNA polymerase (ϕ29DP) were purchased from MBI Fermentas (Burlington, Canada). γ-[³²P]ATP was purchased from PerkinElmer. All other chemicals were purchased from Sigma-Aldrich (Oakville, Canada) and used without further purification.

### Preparation of Circular DNA Templates (CDT)

Phosphorylated linear DNA (300 pmol) was first mixed with a DNA primer (DP; 400 pmol) in 50 μL of H₂O, heated to 90° C. for 1 min, cooled at room temperature for 15 min. To this mixture were added 10 μL of 10×T4 DNA ligase buffer and 10 U (units) of T4 DNA ligase, and water to a total volume of 100 μL. The resultant mixture was incubated at 4° C. overnight. The ligated CDT molecules were concentrated by standard ethanol precipitation and purified by 10% dPAGE.

### Preparation of Radioactive DNA Molecules

Radioactive DNA oligonucleotides was labelled with γ-[³²P]ATP at the 5′ end using T4 polynucleotide kinase according to the manufacturer's protocol. The typical procedure was: A reaction mixture (100 μL) was made to contain 1× Reaction Buffer A, 2 μM DNA oligonucleotide, 10 μCi [γ-³²P]ATP and 0.1 U/μL PNK. The mixture was incubated at 37° C. for 30 min. To ensure that all DNA molecules contained the 5′ phosphate required for ligation, PNK mediated end-labelling solution was further incubated with 10 mM non-radioactive ATP at 37° C. for 20 min before heating at 90° C. for 10 min to deactivate PNK. The radioactive DNA molecules were then concentrated by standard ethanol precipitation and purified by 10% dPAGE.

### RCA Reactions

Typical RCA reactions were performed as follows: A reaction mixture (50 μL) was made that contained 1×RCA reaction buffer, 100 nM DP, 50 nM CDT, 1 mM dNTPs and 0.2 U/μL ϕ29DP. The mixture was incubated at 37° C. for 60 min before heating at 65° C. for 10 min to deactivate ϕ29DP.

### Gel Images

The autoradiogram images of gels were obtained using a Typhoon 9200 variable mode imager (GE healthcare) and analyzed using Image Quant software (Molecular Dynamics).

### Electrophoresis Mobility Shift Assay (EMSA; FIG. 2b)

Preparation of RP and MRP:

The RP used in this experiment was prepared using DP1 and CDT1 according to RCA reactions. The sequence of MRP that is complementary to CDT1 is provided in Table 1.

Quantification of RP:

RP was quantified using a modified digestion method previously described [19]. The typical procedure was: 1 μL of the above RP was first mixed with 5 μL of 100 μM DT1, heated at 90° C. for 5 min, cooled at room temperature (˜22° C.) for 15 min. This was followed by the addition of 1 μL of 10× Fast digestion buffer and 3 μL of EcoRV. The reaction mixture was then incubated at 37° C. for 24 h. The digestion product was mixed with 2 μL of 1 μM DC1 (a DNA control for gel analysis) and 10 μL of 2× denaturing gel loading buffer. The mixture was then run on a 10% dPAGE gel and stained with 1×SYBR Gold before scanning. The concentration of RP was calculated according to the fluorescence ratio between the monomeric DNA band and the DC1 band.

EMSA Procedure:

The DNA hybridization was performed in 10 μL of 1×RCA reaction buffer containing 100 nM of ³²P labelled DDS (a variant of RDS1 in which the embedded adenosine ribonucleotide was replaced by adenosine deoxyribonucleotide), and 200 nM ³²P labelled CDT1. The mixture was heated at 90° C. for 1 min and cooled to RT for 15 min before the addition of RP (corresponding to ˜10 pmol of MRP) or MRP (10 pmol). The reaction solution was incubated at room temperature for 30 min before analysis by 10% native PAGE.

Cleavage Reaction with Doubly Labeled RDS (FIG. 3b)

Preparation of RP:

The RP used in this experiment was prepared using DP1 and CDT1 according to RCA reactions.

Cleavage and Nucleolytic Trimming Reaction Procedure:

Six 25-μL reactions were conducted in this experiment (lanes 1-6), all of which contained 1×RCA reaction buffer and 0.5 μM doubly labeled RDS2. The full reaction (lane 6) also contained 1 μM CDT2, 5 μL of RP made above, 0.5 U/μL PNK and 0.5 U/μL ϕ29DP; however, one or more of these reactions components were omitted in the control reactions as indicated in FIG. 3b (lanes 1-5). RDS2, CDT2, and RP were first incubated at 37° C. for 10 min, then incubated with PNK at 37° C. for 30 min. The resultant mixture was heated at 75° C. for 15 min, and then incubated with ϕ29DP at 37° C. for 30 min. The DNA molecules in the reaction mixtures were subsequently subjected to 10% dPAGE analysis.

### Colorimetric Reporting of Successful RCA Reaction (FIG. 3c)

Preparation of RP:

The RP used in this experiment was prepared using DP1 and CDT1 according to RCA reactions.

The Reporting Assay Procedure:

The reaction was performed in 50 μL of 1×RCA reaction buffer containing 50 nM RDS2, 100 nM CDT2, 1 mM dNTPs, 1 μL of the RP prepared above, 2 μM hemin, 0.2 U/μL PNK and 0.2 U/μL ϕ29DP. The reaction mixture was incubated at 37° C. for 30 min before heating at 65° C. for 10 min. After cooling to room temperature, 2 μL of ABTS (50 mM, final concentration) and 1 μL of H₂O₂ (8.8 mM, final concentration) were added, and the colorimetric result was recorded immediately using a digital camera.

qPCR Procedure

The cDNA samples were prepared by using a reverse transcription (RT) reaction with a qScript™ microRNA cDNA Synthesis Kit (Quanta Biosciences) according to manufacturer instructions. The 20 μL of cDNA synthesis reaction was composed of a 200 ng total small RNA sample, or the desired amount of synthetic miR-21 as the standards. The cDNA products were stored at −20° C. qRT-PCR was performed using 200 nM of each PerfeCTa microRNA Assay Primer and PerfeCTa Universal PCR Primer along with the PerfeCTa SYBR Green SuperMix product on a BioRad CFX96 qPCR system. The 50-μL qRT-PCR solution contained 20 ng total small RNA sample, or miR-21 with desired amounts of RNA. The conditions were as follows: an initial incubation at 95° C. for 2 min, followed by 40 cycles of 94° C. for 5 s, 60° C. for 15 s and 70° C. for 15 s. All standard dilutions and unknown samples were assayed in triplicate. Absolute quantification of miR-21 in cells was achieved by comparing the CT values of the test samples to a standard curve.

### Example 2. Detection of miRNAs and Bacteria Using DFA

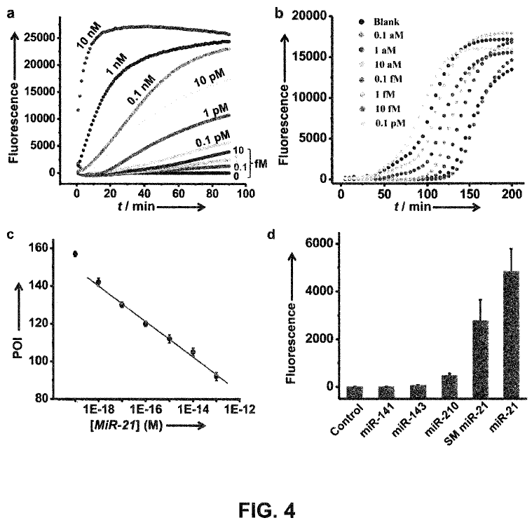

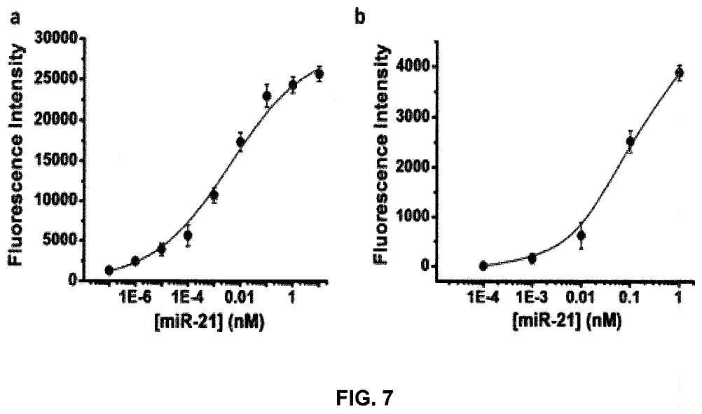

The potential analytical utility of DFA was investigated through two experiments designed to detect a microRNA (miRNA) and E. coli. MiRNAs are a group of small regulatory RNAs^{[14]} that can be used as biomarkers for cancer diagnosis and prognosis.^{[15]} A DFA system was designed to detect miR-21, which is overexpressed in cancer cells.^{[16]} The reaction scheme is identical to FIG. 1 except that miR-21 is used to substitute DR The RP synthesis in response to varying concentrations of miR-21 was monitored in real time by measuring the fluorescence emission of SYBR Gold, a dye that produces enhanced fluorescence upon binding to single-stranded DNA. The limit of detection was found to vary with the DFA reaction time. At 90 minutes, the system can achieve a limit of detection (LOD) of 0.1 fM (FIG. 4a; FIG. 7a). At this reaction time, the rate of signal response at ultralow miRNA concentrations (such as 0.1 fM) was small and did not exhibit exponential amplification behaviour observed for high miRNA concentrations (such as 10 nM). When the reaction time was extended to 200 minutes, DFA reactions at all concentrations as well as the background reaction (no miRNA was provided) exhibited exponential amplification (FIG. 4b). The point of inflection (POI) was then calculated for the reaction profiles in FIG. 4b, and demonstrated a clear logarithmic dependence on target concentration, (FIG. 4c), with a LOD of 1 aM. Based on our sample volume of 50 μL, this corresponds to a LOD of 50 ymol, or 30 molecules. Several previous studies have reported other exponential amplification strategies for miRNA detection with excellent detection sensitivity, with LODs ranging between 10 aM and 0.1 μM (see Table 2 for detailed comparisons).^{[4c,17]} The DFA strategy described herein offers much better (LODs of 1 fM-0.1 μM)^{[17a-d]} or similar (10 aM)^{[17e]} sensitivity. It is worth noting the LOD of the DFA system is six orders of magnitude lower than that of standard RCA (LOD of 1 μM, FIG. 7b).

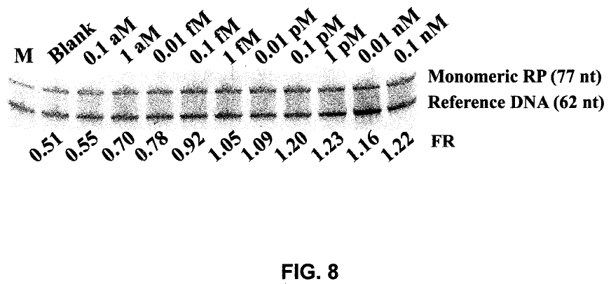

To provide additional evidence for the successful DFA reactions at ultralow miRNA concentrations, an experiment was performed where gel electrophoresis was used to analyze the RCA product using a previously reported method.^{[2f]} Briefly, the RP generated from 200-minute DFA reactions initiated by progressively decreasing miRNA concentrations (10-fold serial decrease from 0.1 nM to 0.1 aM) were digested into monomers and then analyzed by 10% denaturing polyacrylamide gel electrophoresis (FIG. 8). The result obtained is consistent with the data from fluorescence measurements.

The method was also employed to measure the level of miR-21 in total small RNA prepared from the human breast cancer cell line MCF-7 and normal mammary epithelial cell line MCF-10A. The absolute amount of miR-21 found in MCF-7 and MCF-10A cells was found to be 2.6×10⁶ copies/ng RNA (or 4000 copies/cell) and 2.6×10⁵ copies/ng RNA (or 90 copies/cell), respectively. These values are comparable to those obtained using qPCR (Table 3).

The selectivity of the DFA method for miRNA detection was also examined by comparing the signal response in the presence of miR-21 (the intended miRNA), miR-141, miR-143, and miR-210 (which have different sequences from miR-21), and SM miR-21 (differing from miR-21 by one nucleotide). As shown in FIG. 4d, high levels of selectivity were observed for miR-21 against miR-141, miR-143, miR-210 (>99% confidence using the student t-test). A detectable difference was also observed between miR-21 and SM miR-21 (FIG. 4d); however the student t-test analysis indicates that the selectivity is statistically less significant (<90% confidence).

In the second experiment, a DFA reaction for detection of E. coli was designed to take advantage of EC1, a previously reported RCD that can only be activated by E. coli.^{[18]} As illustrated in FIG. 5a, three DNA molecules—CDT, RDS and EC1—were used to set up the specific DFA for this reaction. In addition, the sequence of RDS was carefully designed so that this molecule could be cleaved by both EC1 and MgZ. The starting point of this particular DFA reaction was the cleavage of RDS in complex with CDT by EC1 in the presence of E. coli. This was followed by nucleotide trimming by ϕ29DP to produce the Complex I needed for the DFA reaction.

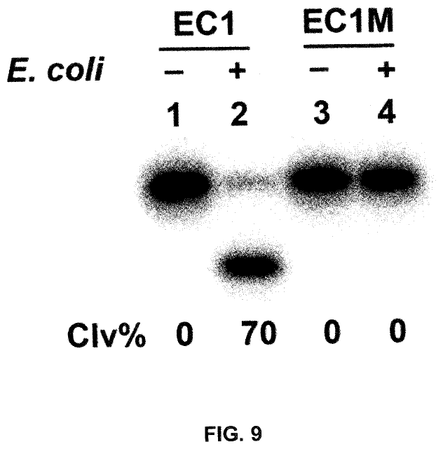

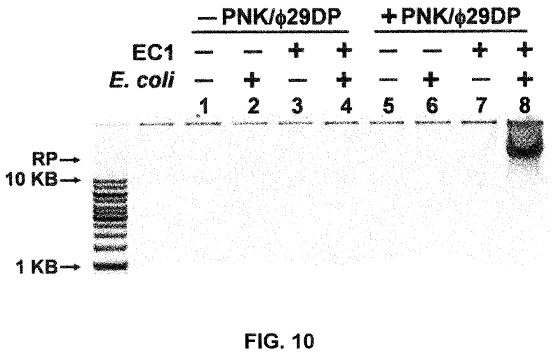

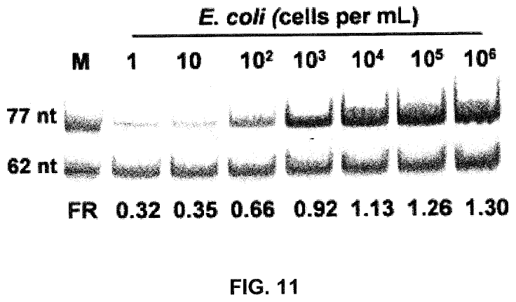

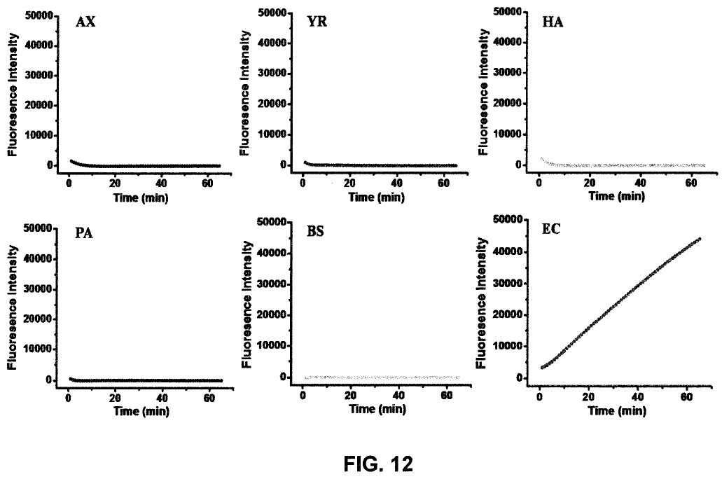

It was observed that EC1 was indeed able to cleave RDS in the presence of E. coli (FIG. 9). More importantly, it was able to trigger DFA, based on the appearance of the RP band on the agarose gel (FIG. 10). The DFA reaction was also subjected to quantitative analysis either via gel electrophoresis (FIG. 11) or simply via fluorescence measurement in the presence of SYBR Gold (FIG. 5b). The LOD by the fluorescence measurement was 10 cells/mL using a 60 min reaction time. This represents a 1000-fold improvement in LOD over the DNAzyme assay without signal amplification.^{[17]} To evaluate the detection specificity, three other gram-negative bacteria and two gram-positive bacteria were tested. The fluorescent results indicated that these bacteria were not able to initiate the DFA reaction (FIG. 12).

### Methods and Experimental Details

DFA Reaction for the Detection of miR-21 (FIG. 4)

Cell Culture and miRNAs Extraction:

The adherent breast cancer cell line MCF-7 was cultured in α-MEM media (GIBCO) with 10% fetal bovine serum (Invitrogen). MCF-10A (mammary epithelial cell line) was cultured in D-MEM medium with 5% (v/v) horse serum, 10 μg/mL human insulin, 10 ng/mL epidermal growth factor, 500 ng/mL hydrocortisone and 10 μM isoproterenol. These cells were cultured at 37° C. in a humidified atmosphere containing 5% CO₂. miRNAs (<200 nt) were extracted and purified using the E.Z.N.A®miRNA Kit according to the manufacturer's protocol. The RNA quantity was determined by measuring optical density at 260 nm using a NanoVue™ Plus spectrophotometer.

Detection Sensitivity (FIG. 4a):

The reaction was performed in 50 μL of 1×RCA reaction buffer containing 300 nM CDT1, 200 nM RDS1, 1 mM dNTPs, 0.2 U/μL PNK, 0.2 U/μL ϕ29DP, 1×SYBR Gold and different amounts of miR-21 targets as indicated in FIG. 4a. These reactions were carried out in a BioRad CFX96 qPCR system set to a constant temperature of 37° C., and the fluorescence intensity was recorded in 1-min intervals. For the detection of low concentrations of miR-21 targets as indicated in FIG. 4b, the reaction was performed under the same conditions as mentioned above except for the use of 5-min intervals for recording the fluorescence signal and a reaction time of 200 min.

Detection Specificity (FIG. 4b):

The reaction was performed in 50 μL of 1×RCA reaction buffer containing 300 nM CDT1, 200 nM RDS1, 1 mM dNTPs, 0.2 U/μL PNK, 0.2 U/μL ϕ29DP, 1×SYBR Gold and 0.1 μM miR-21 or unintended targets (including SM miR-21, miR-141, miR-143 and miR-210) as indicated in FIG. 4b. These reactions were carried out in a BioRad CFX96 qPCR system set to a constant temperature of 37° C., and the fluorescence intensity was recorded in 1-min intervals.

DFA Reaction for E. coli Detection (FIG. 5)

Bacterial sample preparation: E. coli K12 was grown onto a Luria Broth (LB) agar plate for 12 h at 37° C. A single colony was taken, inoculated into 2 mL of LB and grown at 37° C. with shaking at 250 rpm until the culture reached an OD₆₀₀ of ˜1. 1 mL of this culture was centrifuged at 13,000 g for 10 min at 4° C. The cell pellet was suspended in 100 μL of 1× reaction buffer (1×RB, 50 mM HEPES buffer, 150 mM NaCl, 15 mM MgCl₂, pH 7.5). The E. coli cells were sonicated for 30 s and put on ice for 5 min. This process was repeated five times. The cell suspension was then centrifuged at 13,000 g for 10 min at 4° C. The obtained crude intracellular mixture (CIM) in the supernatant was used for the following experiment.

Real-Time Monitoring of DFA at Various E. coli Concentrations (FIG. 5b):

The cleavage reaction was first carried out in 20 μL of 1×RB containing 300 nM CDT1, 200 nM RDS3, 10 μM EC1, 0.5 U/μL PNK, and 5 μL of CIM (prepared from different numbers of E. coli cells). The above mixture was incubated at 37° C. for 60 min. Then 0.2 U/μL ϕ29DP, 1 mM dNTPs, 1×SYBR Gold and 5 μL of 10×RCA reaction buffer were added. These reactions were monitored using a BioRad CFX96 qPCR system set to a constant temperature of 37° C., and the fluorescence intensity was recorded in 1-min intervals.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1

| Name of DNA oligonucleotide | Sequence(5′-3′) | SEQ ID NO: |
|---|---|---|
| Precursor of circular DNA | | |
| template (CDT) | | |
| CDT1 (77 nt) | TCT GAT AAG CTA CCT AGC ATA GCC TCC CAA | SEQ ID NO: 1 |
| | AAT ATC CTA TAT TTC GGC CCC GAC CTG GTT | |
| | CGA TAT CTC AAC ATC AG | |
| CDT2 (51 nt) | ATT CGT GTG AGA AAA CCC AAC CCG CCC | SEQ ID NO: 2 |
| | TAC CCA AAA GAT ATC GTC AGA TGA | |
| DNA primer for ligation (DP) | | |
| DP1 (22 nt) | TAG CTT ATC AGA CTG ATG TTG A | SEQ ID NO: 3 |
| DP2 (20 nt) | CTC ACA CGA ATT CAT CTG AC | SEQ ID NO: 4 |
| RNA-Containing DNA substrate | | |
| (RDS) | | |
| RDS1 (62 nt) | TAG CTT ATC AGA CTG ATG TTG ATT TTT TTT | SEQ ID NO: 5 |
| | TTT TAC TCT TCC TAG CT rA TGG TTC GAT | |
| | CAA GA/3InvdT/ | |
| RDS2 (56 nt) | CAC ACG AAT TCA TCT GTT TTT TTT TTT TAC | SEQ ID NO: 6 |
| | TCT TCC TAG CTrA TGG TTC GAT CAA | |
| | GA/3InvdT/ | |
| RDS3 (74 nt) | TAG CTT ATC AGA CTG ATG TTG ATT TTT TTT | SEQ ID NO: 7 |
| | TTT TAC TCT TCC TAG CF rA QGG TTC GAT | |
| | CAA GAT CTC TCT CTC TC/3InvdT/ | |
| DDS (62 nt) | TAG CTT ATC AGA CTG ATG TTG ATT TTT TTT | SEQ ID NO: 8 |
| | TTT TAC TCT TCC TAG CTA TGG TTC GAT CAA | |
| | GA | |
| Mg²⁺-dependent DNAzyme | | |
| MgZ | GAA CCA GGT CGG GGC CGA AAT ATA GGA | SEQ ID NO: 9 |
| | TAT TTT GGG AGG CTA TGC TAG G/3invdT/ | |
| E. coli-dependent DNAzyme | | |
| EC1 | GAT GTG CGT TGT CGA GAC CTG CGA CCG | SEQ ID NO: 10 |
| | GAA CAC TAC ACT GTG TGG GGA TGG ATT | |
| | TCT TTA CAG TTG TGT G/3InvdT/ | |
| EC1M | GAT GTG CGT AAA GCT CAC CTG CGA CCG | SEQ ID NO: 11 |
| | GAA CAC TAC TGA CAC TGG GGA TGG ATT | |
| | TCT TTA CAG TTG TGT G/3InvdT/ | |
| miRNA target | | |
| miR-21 | UAG CUU AUC AGA CUG AUG UUG A | SEQ ID NO: 12 |
| miR-210 | CUG UGC GUG UGA CAG CGG CUG A | SEQ ID NO: 13 |
| miR-141 | UAA CAC UGU CUG GUA AAG AUG G | SEQ ID NO: 14 |
| miR-143 | UGA GAU GAA GCA CUG UAG CUC A | SEQ ID NO: 15 |
| SM miR-21 (with single mutation) | UAG CUU AUC AGA CUG AUG AUG A | SEQ ID NO: 16 |
| DNA template for digestion (DT) | | |
| DT1 | TGG TTC GAT ATC TCA ACA TCA | SEQ ID NO: 17 |
| DNA control for gel analysis (DC1) | | |
| DC1 | GAC GCG GGA TCC GAC GTT TTT TTT TTT TAC | SEQ ID NO: 18 |
| | TCT TCC TAG CTA TGG TTC GAT CAA GAT CAA | |
| | GA | |
| Monomeric RCA Product | | |
| MRP | ATC GAA CCA GGT CGG GGC CGA AAT ATA | SEQ ID NO: 19 |
| | GGA TAT TTT GGG AGG CTA TGC TAG GTA | |
| | GCT TAT CAG ACT GAT GTT GAG AT | |

rA = adenine ribonucleotide; F = fluorescent moiety; Q = quencher, 3InvdT = 3′ inverted dT

TABLE 2
Isothermal amplification miRNA detection
methods using fluorescence.

| Method | Test time | Sensitivity |
|---|---|---|
| Hairpin probe-RCA | 5 h (hairpin probe reaction - | 10 fM |
| (HP-RCA) [20] | 1 h; RCA - 4 h) | (35° C.) |
| Dumbbell probe-RCA | 8 h (ligation - 2 h; | 1 fM |
| (DP-RCA) [21] | RCA - 6 h) | (30° C.) |
| Branched-RCA (BRCA) | 8.3 h (ligation - 2.1 h; | 10 fM |
| [22] | BRCA 6.2 h) | (30° C.) |
| Ramification | 2.4 h (reverse transcription - | 1 fM |
| amplification (RAM) | 0.8 h; ligation - 0.9; | (65° C.) |
| [23] | RAM - 0.7 h) | |
| Loop-mediated | 2 h | 0.1 pM |
| isothermal amplification | | (55° C.) |
| (LAMP) [24] | | |
| Exponential | 38 min | 10 aM |
| amplification reaction | | (55° C.) |
| (EXPAR) [25] | | |
| DNAzyme feedback | 200 min | 1 aM |
| amplification (DFA) | | (37° C.) |

TABLE 3
Quantification of miR-21 via DFA and qPCR methods.

| | Copies × 10⁵/ng RNA | | Copies/cell | |
|---|---|---|---|---|
| Cell Line | DFA | qPCR | DFA | qPCR |
| MCF-7 | 26 ± 10 | 18 ± 6 | 4000 ± 208 | 3168 ± 101 |
| MCF-10A | 2.6 ± 0.4 | 3 ± 0.2 | 90 ± 15 | 111 ± 8 |

### REFERENCES

[1] a) R. K. Saiki, S. Scharf, F. Faloona, K. B. Mullis, G. T. Horn, H. A. Erlich, N. Arnheim, Science 1985, 230, 1350-1354; b) A. Niemz, T. M. Ferguson, D. S. Boyle, Trends Biotechnol. 2011, 29, 240-250; c) Y. Zhao, F. Chen, Q. Li, L. Wang, C. Fan, Chem. Rev. 2015, 115, 12491-12545.
[2] a) A. Fire, S. Q. Xu, Proc. Natl. Acad. Sci. USA 1995, 92, 4641-4645; b) D. Liu, S. L. Daubendiek, M. A. Zillman, K. Ryan, E. T. Kool, J. Am. Chem. Soc. 1996, 118, 1587-1594; c) W. Zhao, M. M. Ali, M. A. Brook, Y. Li, Angew. Chem. Int. Ed. 2008, 47, 6330-6337; Angew. Chem. 2008, 120, 6428-6436; d) M. M. Ali, F. Li, Z. Zhang, K. Zhang, D. K. Kang, J. A. Ankrum, X. C. Le, W. Zhao, Chem. Soc. Rev. 2014, 43, 3324-3341; e) S. A. McManus, Y. Li, J. Am. Chem. Soc. 2013, 135, 7181-7186; f) M. Liu, W. Zhang, Q. Zhang, J. D. Brennan, Y. Li, Angew. Chem. Int. Ed. 2015, 54, 9637-9641; Angew. Chem. 2015, 127, 9773-9777; g) M. Liu, Q. Zhang, Z. Li, J. Gu, J. D. Brennan, Y. Li, Nat. Commun. 2016, 7, 12704.
[3] S. Kamtekar, A. J. Berman, J. Wang, J. M. Lázaro, M. de Vega, L. Blanco, M. Salas, T. A. Steitz, Mol. Cell 2004, 16, 609-618.
[4] a) F. B. Dean, S. Hosono, L. Fang, X. Wu, A. F. Faruqi, P. Bray-Ward, Z. Sun, Q. Zong, Y. Du, J. Du, M. Driscoll, W. Song, S. F. Kingsmore, M. Egholm, R. S. Lasken, Proc. Natl. Acad. Sci. USA. 2002, 99, 5261-5266; b) P. M. Lizardi, X. Huang, Z. Zhu, P. Bray-Ward, D. C. Thomas, D. C. Ward, Nat. Genet. 1998, 19, 225-232; c) Y. Cheng, X. Zhang, Z. Li, X. Jiao, Y. Wang, Y. Zhang, Angew. Chem. Int. Ed. 2009, 48, 3268-3272; Angew. Chem. 2009, 121, 3318-3322.
[5] a) T. Murakami, J. Sumaoka, M. Komiyama, Nucleic Acids Res. 2009, 37, e19; b) T. Murakami, J. Sumaoka, M. Komiyama, Nucleic Acids Res. 2011, 40, e22; c) F. Dahl, J. Baner, M. Gullberg, M. Mendel-Hartvig, U. Landegren, M. Nilsson, Proc. Natl. Acad. Sci. USA. 2004, 101, 4548-4553; d) F. Wang, C. Lu, X. Liu, L. Freage, I. Willner, Anal. Chem. 2014, 86, 1614-1621.
[6] a) L. C. Bock, L. C. Griffin, J. A. Latham, E. H. Vermaas, J. J. Toole, Nature 1992, 355, 564-566; b) A. D. Ellington, J. W. Szostak, Nature 1992, 355, 850-852; c) R. R. Breaker, Nat. Biotechnol. 1997, 15, 427-431; d) K. Schlosser, Y. Li, Chem. Biol. 2009, 16, 311-322; e) S. K. Silverman, Angew. Chem. Int. Ed. 2010, 49, 7180-7201; Angew. Chem. 2010, 122, 7336-7359; f) N. Carmi, S. R. Balkhi, R. R. Breaker, Proc. Natl. Acad. Sci. U.S.A. 1998, 95, 2233-2237; g) D. J. Chinnapen, D. Sen, Proc. Natl. Acad. Sci. U.S.A. 2004, 101, 65-69; h) M. Chandra, A. Sachdeva, S. K. Silverman, Nat. Chem. Biol. 2009, 5, 718-720.
[7] a) A. D. Ellington, J. W. Szostak, Nature 1990, 346, 818-822; b) C. Tuerk, L. Gold, Science 1990, 249, 505-510; c) D. L. Robertson, G. F. Joyce, Nature 1990, 344, 467-468.
[8] a) R. R. Breaker, G. F. Joyce, Chem. Biol. 1994, 1, 223-229; b) S. W. Santoro, G. F. Joyce, Proc. Natl. Acad. Sci. U.S.A 1997, 94, 4262-4266; c) S. K. Silverman, Nucleic Acids Res. 2005, 33, 6151-6163.
[9] a) D. M. Perrin, T. Garestier, C. Helene, J. Am. Chem. Soc. 2001, 123, 1556-1563; b) S. H. J. Mei, Z. Liu, J. D. Brennan, Y. Li, J. Am. Chem. Soc. 2003, 125, 412-420; c) J. Liu, A. K. Brown, X. Meng, D. M. Cropek, J. D. Istok, D. B. Watson, Y. Lu, Proc. Natl. Acad. Sci. U.S.A. 2007, 104, 2056-2061; d) P. J. Huang, M. Vazin, J. Liu, Anal. Chem. 2014, 86, 9993-9999; e) S. F. Torabi, P. Wu, C. E. McGhee, L. Chen, K. Hwang, N. Zheng, J. Cheng, Y. Lu, Proc. Natl. Acad. Sci. U.S.A. 2015, 112, 5903-5908; f) Z. Shen, Z. Wu, D. Chang, W. Zhang, K. Tram, C. Lee, P. Kim, B. J. Salena, Y. Li, Angew. Chem. Int. Ed. 2016, 55, 2431-2434; Angew. Chem. 2016, 128, 2477-2480.
[10] a) N. Navani, Y. Li, Curr Opin Chem Biol. 2006, 10, 272-281; b) J. Liu, Y. Lu, Chem. Rev. 2009, 109, 1948-1998; c) F. Wang, C. Lu, I. Willner, Chem. Rev. 2014, 114, 2881-2941.
[11] a) J. Li, Y. Lu, J. Am. Chem. Soc. 2000, 122, 10466-10467; b) Y. Xiang, Y. Lu, Nat. Chem. 2011, 3, 697-703; c) K. Hwang, P. Wu, T. Kim, L. Lei, S. Tian, Y. Wang, Y. Lu, Angew. Chem. Int. Ed. 2014, 53, 13798-13802; d) P. J. Huang, J. Liu, Anal. Chem. 2014, 86, 5999-6005; e) K. Tram, P. Kanda, B. J. Salena, S. Huan, Y. Li, Angew. Chem. Int. Ed. 2014, 53, 12799-12802; Angew. Chem. 2014, 126, 13013-13016; f) Z. Shen, Z. Wu, D. Chang, W. Zhang, K. Tram, C. Lee, P. Kim, B. J. Salena, Y. Li, Angew. Chem. Int. Ed. 2016, 55, 2431-2434; Angew. Chem. 2016, 128, 2477-2480; g) W. Zhang, Q. Feng, D. Chang, K. Tram, Y. Li, Methods. 2016, 106, 66-75.
[12] W. Chiuman, Y. Li, PLoS One 2007, 2, e1224.
[13] a) P. Travascio, Y. Li, D. Sen, 1998, Chem. Biol. 5, 505-517; b) P. Travascio, P. K. Witting, A. G. Mauk, D. Sen, J. Am. Chem. Soc. 2001, 123, 1337-1348; c) Z. Cheglakov, Y. Weizmann, B. Basnar, I. Willner, Org. Biomol. Chem. 2007, 5, 223-225.
[14] a) L. He, G. J. Hannon, Nat. Rev. Genet. 2004, 5, 522-531; b) S. L. Ameres, P. D. Zamore, Nat. Rev. Mol. Cell Biol. 2013, 14, 475-488.
[15] a) B. M. Ryan, A. I. Robles, C. C. Harris, Nat. Rev. Cancer 2010, 10, 389-402; b) H. Dong, J. Lei, L. Ding, Y. Wen, H. Ju, X. Zhang, Chem. Rev. 2013, 113, 6207-6233; c) J. Li, S. Tan, R. Kooger, C. Zhang, Y. Zhang, Chem. Soc. Rev. 2014, 43, 506-517.
[16] A. M. Krichevsky, G. Gabriely, J. Cell. Mol. Med. 2009, 13, 39-53.
[17] a) Y. Li, L. Liang, C. Y. Zhang, Anal. Chem. 2013, 85, 11174-11179; b) Y. T. Zhou, Q. Huang, J. M. Gao, J. X. Lu, X. Z. Shen, C. H. Fan, Nucleic Acids Res. 2010, 38, e156; c) B. Yao, J. Li, H. Huang, C. Sun, Z. Wang, Y. Fan, Q. Chang, S. Li, J. Xi, RNA 2009, 15, 1787-1794; d) C. P. Li, Z. P. Li, H. X. Jia, J. L. Yan, Chem. Commun. 2011, 47, 2595-2597; e) H. Jia, Z. Li, C. Li, Y. Cheng, Angew. Chem. Int. Ed. 2010, 49, 5498-5501; Angew. Chem. 2010, 122, 5630-5633
[18] a) M. M. Ali, S. D. Aguirre, H. Lazim, Y. Li, Angew. Chem. Int. Ed. 2011, 50, 3751-3754; Angew. Chem. 2011, 123, 3835-3838; b) S. D. Aguirre, M. M. Ali, B. J. Salena, Y. Li, Biomolecules 2013, 3, 563-577.
[19] M. Liu, C. Y. Hui, Q. Zhang, J. Gu, B. Kannan, S. Jahanshahi-Anbuhi, C. D. M. Filipe, J. D. Brennan, Y. Li, Angew. Chem. Int. Ed. 2016, 55, 2709-2713; Angew. Chem. 2016, 128, 2759-2763.
[20] Y. Li, L. Liang, C. Y. Zhang, Anal. Chem. 2013, 85, 11174-11179.
[21] Y. T. Zhou, Q. Huang, J. M. Gao, J. X. Lu, X. Z. Shen, C. H. Fan, Nucleic Acids Res. 2010, 38, e156.
[22] Y. Cheng, X. Zhang, Z. Li, X. Jiao, Y. Wang, Y. Zhang, Angew. Chem. Int. Ed. 2009, 48, 3268-3272; Angew. Chem. 2009, 121, 3318-3322.
[23] B. Yao, J. Li, H. Huang, C. Sun, Z. Wang, Y. Fan, Q. Chang, S. Li, J. Xi, RNA 2009, 15, 1787-1794.
[24] C. P. Li, Z. P. Li, H. X. Jia, J. L. Yan, Chem. Commun. 2011, 47, 2595-2597.
[25] H. Jia, Z. Li, C. Li, Y. Cheng, Angew. Chem. Int. Ed. 2010, 49, 5498-5501; Angew. Chem. 2010, 122, 5630-5633.
[26] He S, Qu L, Shen Z, Tan Y, Zeng M, Liu F, Jiang Y, Li Y. “Highly specific recognition of breast tumors by an RNA-cleaving fluorogenic DNAzyme probe.” Anal Chem. 2015 Jan. 6; 87(1):569-77.

1. A method of detecting a target analyte in a sample, the method comprising:
combining the sample with a first circular DNA template comprising a region encoding an antisense ribonucleotide-cleaving DNAzyme (RNA-cleaving DNAzyme), wherein the first circular DNA template is amplified by rolling circle amplification in the presence of the target analyte to produce a first amplification product comprising the RNA-cleaving DNAzyme;
contacting the first amplification product comprising the RNA-cleaving DNAzyme and a substrate complex comprising a ribonucleotide-containing DNA sequence (RDS) nucleic acid molecule annealed to a second circular DNA template, the second circular DNA template comprising a region encoding an antisense DNAzyme and a region complimentary to the 5′ end of the RDS nucleic acid molecule, wherein the RNA-cleaving DNAzyme acts on the substrate complex to produce a 5′ cleavage fragment comprising a 5′ region annealed to the second circular DNA template and a 3′ cleavage fragment;
amplifying the second circular DNA template by rolling circle amplification primed by a 3′-hydroxyl end of the 5′ region annealed to the second circular DNA template to produce a second amplification product comprising the DNAzyme; and
detecting an increase in the first amplification product and/or second amplification product thereby detecting the presence of the target analyte in the sample.

2. The method of claim 1, wherein the target analyte is a target nucleic acid molecule that binds to the first circular DNA template and rolling circle amplification of the first circular DNA template is primed by a 3′-hydroxyl end of the target nucleic acid molecule that binds to the first circular DNA template.

3. The method of claim 1, wherein the target analyte activates an exogenous RNA-cleaving DNAzyme that binds to a nucleic acid molecule annealed to the first circular DNA template comprising one or more RDS sequences to produce a 5′ cleavage product comprising a 5′ region annealed to the first circular DNA template, wherein rolling circle amplification of the first circular DNA template is primed by a 3′-hydroxyl end of the 5′ region annealed to the first circular DNA template.

4. The method of claim 3, wherein the nucleic acid molecule annealed to the first circular DNA template comprises a first RDS sequence that is cleaved by the exogenous RNA-cleaving DNAzyme and a second RDS sequence that is cleaved by an RNA-cleaving DNAzyme encoded by the second circular template, optionally wherein the exogenous RNA-cleaving DNAzyme is EC1.

5. The method of claim 1, wherein the target analyte binds to a recognition moiety that directly or indirectly triggers rolling circle amplification of the first circular DNA template.

6. The method of claim 1, wherein the DNAzyme encoded by the second amplification product acts on a substrate to produce a detectable signal, optionally wherein the DNAzyme is PW17.

7. The method of claim 1, wherein the DNAzyme encoded by the second amplification product is an RNA-cleaving DNAzyme, optionally the same RNA-cleaving DNAzyme that is encoded by the first amplification product and the RNA-cleaving DNAzyme on the second amplification product acts on the RDS nucleic acid molecule on the substrate complex to produce the 5′ cleavage fragment and the 3′ cleavage fragment.

8. The method of claim 1, wherein the 3′ end of the RDS nucleic acid molecule is resistant to exonuclease activity.

9. The method of claim 1, comprising removing unpaired nucleotides from the 5′ cleavage fragment to form the 3′-hydroxyl end of the 5′ region annealed to the second circular DNA template.

10. The method of claim 9, wherein phi 29 DNA polymerase (ϕ29DP) is used for removing unpaired nucleotides from the 5′ cleavage fragment, optionally in the presence of polynucleotide kinase (PNK) and wherein ϕ29DP is used for rolling circle amplification of the first circular DNA template and/or second circular DNA template.

11. (canceled)

12. The method of claim 1, wherein combining the sample with the first circular DNA template, contacting the first amplification product and the substrate complex, and amplifying the second circular DNA template are done at same temperature and/or in the same reaction vessel.

13.-18. (canceled)

19. A method of amplifying a target sequence of a target nucleic acid molecule in a sample, the method comprising:
combining the sample and a first circular DNA template comprising a region encoding an antisense ribonucleotide-cleaving DNAzyme (RNA-cleaving DNAzyme) and a region complimentary to the target sequence such that a 3′-hydroxyl end of the target nucleic acid molecule anneals to the region complimentary to the target sequence on the circular DNA template;
amplifying the first circular DNA template by rolling circle amplification primed by the 3′-hydroxyl end of the target nucleic acid molecule to produce a first amplification product comprising a RNA-cleaving DNAzyme,
wherein the RNA-cleaving DNAzyme acts on a substrate complex comprising a ribonucleotide-containing DNA sequence (RDS) nucleic acid molecule annealed to a second circular DNA template to produce a 5′ cleavage fragment comprising a 5′ region annealed to the second circular DNA template and a 3′ cleavage fragment; and
amplifying the second circular DNA template by rolling circle amplification primed by a 3′-hydroxyl end of the 5′ region annealed to the second circular DNA template.

20.-21. (canceled)

22. The method of claim 19, wherein amplifying the second circular DNA template by rolling circle amplification produces a second amplification product comprising an RNA-cleaving DNAzyme and the RNA-cleaving DNAzyme acts on the RDS nucleic acid molecule on the substrate complex to produce the 5′ cleavage fragment and the 3′ cleavage fragment.

23.-24.

25. The method of claim 19, wherein the first circular DNA template and the second circular DNA template consist of the same DNA sequence.

26. The method of claim 19, wherein the method comprises combining the RDS nucleic acid molecule and a stoichiometric excess of the first circular DNA template to form a mixture comprising the first circular DNA template and the substrate complex, and combining the sample and the first circular DNA template comprises combining the sample and the mixture, wherein the stoichiometric excess of the first circular DNA template relative to the RDS nucleic acid molecule is at least 3:2.

27.-40. (canceled)

41. The method of claim 19, wherein the sample is treated to render the target nucleic acid molecule single stranded and/or wherein the sample is treated with a restriction enzyme to generate the target nucleic acid molecule.

42. The method of claim 19, further comprising detecting an increase in the first amplification product and/or second amplification product thereby detecting the presence of the target nucleic acid molecule in the sample.

43. (canceled)

44. A kit comprising:
a circular DNA template comprising a region encoding an antisense ribonucleotide-cleaving DNAzyme (RNA-cleaving DNAzyme) and a region complimentary to a target sequence; and
ribonucleotide-containing DNA sequence (RDS) nucleic acid molecule comprising a 5′ region comprising the target sequence and a 3′ region comprising a ribonucleotide that is cleaved by the RNA-cleaving DNAzyme encoded by the circular DNA template.

45.-46. (canceled)

47. The kit of claim 44, wherein the 3′ region of the RDS nucleic acid molecule comprises a cleavage site for an exogenous RNA-cleaving DNAzyme, the exogenous RNA-cleaving enzyme is activated by a target analyte, and the kit further comprises the exogenous RNA-cleaving DNAzyme.

48. The kit of claim 47, further comprising one or more reagents for rolling circle amplification (RCA) of the circular DNA template, optionally wherein the one or more reagents includes ϕ29DP.

49.-51. (canceled)


SEQUENCE LISTING (19 sequences)

SEQ ID NO: 1 - DNA, 77 bp, Artificial Sequence
  Synthetic construct
tctgataagctacctagcatagcctcccaaaatatcctatatttcggccccgacctggttcgatatctcaacatcag

SEQ ID NO: 2 - DNA, 51 bp, Artificial Sequence
  Synthetic construct
attcgtgtgagaaaacccaacccgccctacccaaaagatatcgtcagatga

SEQ ID NO: 3 - DNA, 22 bp, Artificial Sequence
  Synthetic construct
tagcttatcagactgatgttga

SEQ ID NO: 4 - DNA, 20 bp, Artificial Sequence
  Synthetic construct
ctcacacgaattcatctgac

SEQ ID NO: 5 - DNA, 63 bp, Artificial Sequence
  Synthetic construct
  misc_feature (48)..(48): n is rA (Adenine Ribonucleotide)
  misc_feature (63)..(63): n is /3InvdT/
tagcttatcagactgatgttgattttttttttttactcttcctagctntggttcgatcaagan

SEQ ID NO: 6 - DNA, 57 bp, Artificial Sequence
  Synthetic construct
  misc_feature (42)..(42): n is rA (Adenine Ribonucleotide)
  misc_feature (57)..(57): n is /3InvdT/
cacacgaattcatctgttttttttttttactcttcctagctntggttcgatcaagan

SEQ ID NO: 7 - DNA, 73 bp, Artificial Sequence
  Synthetic construct
  misc_feature (46)..(46): Cytosine with Fluorescent Moiety
  misc_feature (47)..(47): Adenine Ribonucleotide
  misc_feature (48)..(48): Guanine with Quench Moiety
  misc_feature (73)..(73): n is /3InvdT/
tagcttatcagactgatgttgattttttttttttactcttcctagcaggttcgatcaagatctctctctctcn

SEQ ID NO: 8 - DNA, 62 bp, Artificial Sequence
  Synthetic construct
tagcttatcagactgatgttgattttttttttttactcttcctagctatggttcgatcaaga

SEQ ID NO: 9 - DNA, 49 bp, Artificial Sequence
  Synthetic construct
gaaccaggtcggggccgaaatataggatattttgggaggctatgctagg

SEQ ID NO: 10 - DNA, 70 bp, Artificial Sequence
  Synthetic construct
gatgtgcgttgtcgagacctgcgaccggaacactacactgtgtggggatggatttctttacagttgtgtg

SEQ ID NO: 11 - DNA, 70 bp, Artificial Sequence
  Synthetic construct
gatgtgcgtaaagctcacctgcgaccggaacactactgacactggggatggatttctttacagttgtgtg

SEQ ID NO: 12 - RNA, 22 nt, Artificial Sequence
  Synthetic construct
uagcuuaucagacugauguuga

SEQ ID NO: 13 - RNA, 22 nt, Artificial Sequence
  Synthetic construct
cugugcgugugacagcggcuga

SEQ ID NO: 14 - RNA, 22 nt, Artificial Sequence
  Synthetic construct
uaacacugucugguaaagaugg

SEQ ID NO: 15 - RNA, 22 nt, Artificial Sequence
  Synthetic construct
ugagaugaagcacuguagcuca

SEQ ID NO: 16 - RNA, 22 nt, Artificial Sequence
  Synthetic construct
uagcuuaucagacugaugauga

SEQ ID NO: 17 - DNA, 21 bp, Artificial Sequence
  Synthetic construct
tggttcgatatctcaacatca

SEQ ID NO: 18 - DNA, 62 bp, Artificial Sequence
  Synthetic construct
gacgcgggatccgacgttttttttttttactcttcctagctatggttcgatcaagatcaaga

SEQ ID NO: 19 - DNA, 77 bp, Artificial Sequence
  Synthetic construct
atcgaaccaggtcggggccgaaatataggatattttgggaggctatgctaggtagcttatcagactgatgttgagat